(19) United States
(12) Patent Application Publication
(10) Pub. No.: US 2023/0257717 A1
(43) Pub. Date: Aug. 17, 2023

(54) CULTURE OF ORGANOIDS

(71) Applicant: GENOME RESEARCH LIMITED, Hinxton (GB)

(72) Inventors: Mathew GARNETT, Hinxton (GB); Stacey PRICE, Cambridge (GB); Hayley FRANCIES, Cambridge (GB)

(21) Appl. No.: 18/012,400

(22) PCT Filed: Jun. 25, 2021
(86) PCT No.: PCT/EP2021/067543

§ 371 Date: Dec. 22, 2022

(30) Foreign Application Priority Data
Jun. 26, 2020 (GB) 2009808.3

(51) Int. Cl. C12N 5/09; G01N 33/50; C12N 15/11; C12N 9/22

(52) U.S. Cl. CPC C12N 5/0693; G01N 33/5011; C12N 15/11; C12N 9/22; C12N 2533/90; C12N 2310/20

(57) ABSTRACT

The present invention provides methods, compositions and kits for use in the culture of organoids in solution. In particular, a method for producing an expanded population of organoids in vitro is provided. The method comprises providing a population of organoid progenitor cells or organoids and culturing the population of organoids in a composition comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml, thereby producing an expanded population of organoids. The invention is particularly useful in the context of high-throughput production of organoids such as e.g. for screening.

### FIELD OF THE INVENTION

The present invention relates to in vitro culture of organoids. Methods, compositions and systems for in vitro culture of organoids in suspension are described, for example for use in high-throughput screens.

### BACKGROUND TO THE INVENTION

Organoids are three-dimensional multicellular constructs derived from primary tissue, embryonic stem cells (ESCs) or induced pluripotent stem cells (iPSCs) which can self-organise and self-renew and replicate at least some of the organ functionalities of the tissue from which they are derived.

Organoids are a valuable and ever maturing resource for the scientific research community. Following the first reports of long term patient derived colon organoid models in 2011 (1) significant advances have been made in the organoid technology and its use. These include intuitive next steps such as increasing the different types of organs that can be modelled in vitro, to the more complex advances in how they can be applied to interrogate a wide variety of biological questions (2). These advances have accelerated both basic and translational research in a wide variety of scientific disciplines from developmental biology to personalised cancer medicine (3).

In cancer research, organoid models are an invaluable pre-clinical tool which complement existing preclinical models, such as 2D cell culture and patient derived xenografts. Organoids add many different benefits to cancer research, most notably the higher success rates of derivation allowing the community to increase and broaden the number of cell models, thereby improving the ability to model the underlying complexity of the disease in vitro.

Organoid models are applicable to nearly all experimental techniques as traditional cell lines, nevertheless, there are considerations to be made when working with these models. In comparison to traditional 2D cell culture techniques the culturing of organoids is more expensive due to the requirement of an extracellular matrix (ECM) such as basement membrane extract (BME, commercially available as Corning™'s Matrigel or Cultrex™ BME), as well as the need for a highly specialised and complex growth medium. The rate of growth of organoids in general is much slower than traditional cell lines and lastly, standard culture protocols grow organoids in small domes of ECM, while applicable for derivation and small expansions, ergonomically this becomes problematic for large expansions. These considerations currently limit their utility in high-throughput phenotypic assays, where multiple models and tens of millions of each organoids are required.

For example, van de Wetering et al. (15) described the establishment of tumour organoid cultures and normal-adjacent organoid cultures from 20 colorectal carcinoma (CRC) patients, which were then used for a proof-of-concept drug screen. Organoid cultured in 5-10 μl BME domes were gently disrupted and plated on BME-coated 384-well plates, where they embedded themselves in the matrix and were left for 6 days with each of an 83 compounds library. In total, approximately 5000 measurements of organoid-drug interactions were measured (including replicates). The authors concluded that tumour organoids are amenable to high-throughput drug screens. However, for truly high-throughput applications such as e.g. a standard genome wide CRISPR library with 100,000 gRNA, transduced at a multiplicity of infection (MOI) of 0.3 and with 100× coverage of each gRNA, a total of 3×10⁷ individual organoids is required per technical replicate. Obtaining and maintaining such large amounts of organoids in small individual domes of ECM is extremely labour and space intensive, and carries a high risk of contamination of the cultures.

Therefore, there remains an unmet need for methods and systems that are amenable to large scale expansions of organoids. The present invention seeks to provide solutions to these needs and provides further related advantages.

### BRIEF DESCRIPTION OF THE INVENTION

The present inventors set out to develop alternative culture techniques to facilitate large expansions of organoids in order to achieve the exceedingly high cell numbers required for high throughput phenotypic screens. The inventors identified low percentage ECM culture of organoids in suspension to be particularly appropriate in supporting large scale organoid expansions. Furthermore, multiple models could be effectively expanded in parallel to efficiently perform pharmacological and genome-wide CRISPR screens on hundreds of organoid models.

Genetic and phenotypic characterisation was performed on a set of six core models to confirm that organoids cultured over an extended period in low ECM conditions did not undergo any significant transformations from their parental counterparts. Specifically, the inventors showed that in the new culture conditions with low ECM, organoid formation and growth is supported, the genetic landscape is stable and phenotypic screening results are consistent with organoid models grown in standard organoid protocols. Therefore, the present invention provides a viable alternative culture method for large-scale organoid expansions. This in turn significantly improves the throughput of drug and whole-genome CRISPR screens in e.g. cancer organoids, with important implications for precision cancer medicine.

Accordingly, in a first aspect the present invention provides a method for producing an expanded population of organoids in vitro comprising: (i) providing a population of organoid progenitor cells or organoids; and (ii) culturing the population of organoids in a composition comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml, thereby producing an expanded population of organoids.

In a second aspect, the present invention provides a composition suitable for expansion of organoids, comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml.

In a third aspect, the present invention provides a method of passaging or changing the medium in an organoid culture, comprising: (i) providing a cell culture comprising a population of organoids and a composition as described herein (such as e.g. according to any embodiment of the second aspect); (ii) centrifuging the cell culture to obtain a pellet comprising the organoid population and a supernatant; (iii) optionally disrupting the organoids; (iv) mixing the (optionally disrupted) organoids with a composition as described herein, thereby producing a passaged or medium-changed organoid culture.

Disrupting the organoids may comprise exposing the pellet comprising the organoids to a proteolytic solution, preferably for between 5 and 10 minutes. Disrupting the organoids may instead or in addition comprise mechanically disrupting the organoids. The method may further comprise centrifuging the composition to obtain a pellet comprising the organoid population and a supernatant. Centrifuging the cell culture may comprise centrifuging the population at 400 g, 500 g, 600 g, 700 g, 800 g or 900 g for about 60 seconds, 90 seconds, 120 seconds, 150 seconds or 180 seconds, preferably at 800 g for 120 seconds.

The method may further comprise dispensing the passaged or medium-changed organoid culture in one or more low adhesion cell culture containers.

Mixing the organoids with a composition as described herein may comprise mixing the organoids with a culture medium and adding a scaffold matrix to the composition comprising the organoids and the culture medium.

In a fourth aspect, the present invention provides a method of screening an organoid or a population of organoids comprising: contacting an organoid or population of organoids with a test compound; and determining the effect of the test compound on the organoids or population of organoids, wherein the organoids or population of organoids were obtained using the methods of the first aspect, and/or wherein the contacting is performed while the organoids are in suspension in a composition as described herein (such as e.g. according to any embodiment of the second aspect).

Screening may comprise performing a drug screen, gene editing screen or RNA interference screen. Advantageously, screening may comprise performing a CRISPR gene editing screen. A gene editing screen may advantageously be a genome-wide gene editing screen.

In a fifth aspect, the present invention provides a kit for the production of expanded populations of organoids comprising a composition as described herein (such as e.g. according to any embodiment of the second aspect) or a culture medium (or equivalent amount of concentrated medium) and a scaffold matrix in relative amounts as described herein.

The kit may further comprise one or more low adherence cell culture containers. The one or more low adherence cell culture containers may include one or more cell culture containers coated with an anti-adhesion coating. The anti-adhesion coating may be a covalently bound hydrogel layer or a covalently bound hydrophobic polymer, such as a hydrophobic fluorinated polymer.

Any of the methods described herein (such as e.g. according to the first or fourth aspect) may comprise preparing an organoid culture by mixing organoids or organoid progenitor cells and a composition comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml.

Any of the methods described herein (such as e.g. according to the first or fourth aspect) may comprise preparing an organoid culture by mixing organoids or organoid progenitor cells and a culture medium and a scaffold matrix, wherein the amounts of scaffold matrix and culture medium are such that the scaffold matrix is present in the resulting composition at a concentration that is equivalent to a concentration of between 2% (v/v) and 18% (v/v) of a complex protein hydrogel having a protein concentration between 12 and 18 mg/ml.

In embodiments of any aspect, the scaffold matrix is present in the composition at a concentration that is equivalent to a concentration of at least 3% (v/v) or at least 4% (v/v) of a complex protein hydrogel having a protein concentration between 12 and 18 mg/ml.

The use of concentrations that are equivalent to a concentration of at least 2.5%, or at least 3% (v/v) (such as e.g. approx. 4% or approx. 5% (v/v)) may be particularly advantageous as lower concentrations may limit the amount of organoids that can be grown in the composition. Indeed, without wishing to be bound by theory, it is believed that all organoids require access to the matrix for suitable growth. The present inventors have found concentrations around 5% (v/v) to strike a good balance in terms of enabling high throughput propagation of organoids in suspension while maintaining the matrix requirements advantageously low.

A concentration of scaffold matrix that is equivalent to a concentration of between 2% (v/v) and 18% (v/v) of a complex protein hydrogel having a protein concentration between 12 and 18 mg/ml may be a concentration that is between 0.02 and 0.225 times the concentration of scaffold matrix usable to culture organoids embedded in domes of the scaffold matrix.

In embodiments of any aspect, the scaffold matrix is present in the composition at a concentration that is equivalent to a concentration of between 3% (v/v) and 15% (v/v) of a complex protein hydrogel having a protein concentration between 12 and 18 mg/ml. In embodiments of any aspect, the scaffold matrix is present in the composition at a concentration that is equivalent to a concentration of between 2.5% (v/v) and 15% (v/v) of a complex protein hydrogel having a protein concentration between 12 and 18 mg/ml.

In embodiments of any aspect, the scaffold matrix is present in the composition at a concentration that is between 0.03 and 0.1875 times the concentration of scaffold matrix usable to culture organoids embedded in domes of the scaffold matrix.

The scaffold matrix may be a complex protein hydrogel. In embodiments, the complex protein hydrogel is present in the composition at a concentration that results in a protein concentration from the complex protein hydrogel of between 0.24 mg/ml and 3.24 mg/ml, preferably between 0.3 and 2.7 mg/ml or between 0.36 and 2.7 mg/ml.

In embodiments, the scaffold matrix is a basement membrane extract, preferably a soluble form of basement membrane purified from Engelbreth-Holm-Swarm (EHS) sarcoma cells, such as Cultrex™ BME, Cultrex™ BME type 3, Cultrex™ BME type 2, or Corning™ Matrigel™. In embodiments, the scaffold matrix is Cultrex™ BME type 3 or Cultrex™ BME type 2. Advantageously, the scaffold matrix may be Cultrex™ BME type 2. Advantageously, the Cultrex™ BME type 3 or Cultrex™ BME type 2 may be present at a concentration of between 2% (v/v) and 18% (v/v), between 3% (v/v) and 18% (v/v), or between 3% (v/v) and 15% (v/v).

The scaffold matrix may be present in the composition at a concentration that is equivalent to a concentration of between 2.5% (v/v) and 18% (v/v), between 3% (v/v) and 18% (v/v), between 4% (v/v) and 18% (v/v), between 3% (v/v) and 15% (v/v), between 4% (v/v) and 15% (v/v), between 4% (v/v) and 12% (v/v), between 5% (v/v) and 10% (v/v), or about 5% (v/v) of a complex protein hydrogel having a protein concentration between 12 and 18 mg/ml.

The scaffold matrix may be present in the composition at a concentration that is between 0.03 and 0.225, between 0.04 and 0.1875, between 0.03 and 0.1875, between 0.04 and 0.15, between 0.04 and 0.125, between 0.05 and 0.125, or about 0.05-0.0625 times the concentration of the scaffold matrix that is usable to culture organoids embedded in domes of the scaffold matrix.

The culture medium may be a chemically defined medium. The culture medium advantageously comprises a basal medium, such as Advanced Dulbecco's modified eagle medium (DMEM). The culture medium may comprise a media supplement, such as N2 (Gibco), B-27™ (ThermoFisher). The culture medium may comprise one or more supplements which may include supplements selected from: L-glutamine or substitutes, such as L-alanyl-L-20 glutamine (e.g. Glutamax™), nicotinamide, N-acetylcysteine, buffers, such as HEPES, and antibiotics such as blasticidin or puromycin.

The culture medium may additionally comprise one or more compounds selected from: growth factors (such as epidermal growth factor (EGF), fibroblast growth factor 10 (FGF10)), a TGFβ inhibitor, a non-canonical Wnt signalling potentiator, a BMP inhibitor, hormones (such as e.g. gastrin and/or prostaglandin E2), a canonical Wnt ligand, and a p38 MAPK signalling inhibitor.

In accordance with any aspect, culturing a population or organoids or organoid progenitor cells may comprise maintaining the composition comprising the population of organoids in one or more low adherence cell culture containers. The low adherence cell culture containers may be cell culture containers coated with an anti-adhesion coating. The anti-adhesion coating may be a covalently bound hydrogel layer or a covalently bound hydrophobic polymer, such as a hydrophobic fluorinated polymer.

Culturing the population of organoids or organoid progenitor cells may comprise culturing the population in suspension in the composition.

Any of the methods described herein (such as e.g. according to the first, third or fourth aspect) may comprise maintaining the organoids in culture and expanding them to a population comprising at least 10\hat{ }4, at least 10\hat{ }5, at least 10\hat{ }6 or at least 10\hat{ }7 individual organoids.

Any of the methods described herein (such as e.g. according to the first, third or fourth aspect) may comprise maintaining the organoids in culture for at least 4 weeks, at least 6 weeks, at least 8 weeks, at least 2 months, at least 3 months, at least 4 months, at least 5 months or at least 6 months.

Any of the methods described herein (such as e.g. according to the first, third or fourth aspect) may comprise maintaining the organoids in culture for at least 2 passages, at least 3 passages, at least 4 passages, at least 6 passages, at least 8 passages, at least 10 passages, at least 12 passages or at least 14 passages.

In accordance with any aspect, the organoids may be colon, pancreas, oesophagus, breast, lung, ovary or prostate organoids. In accordance with any aspect, the organoids may be derived from primary tissue, preferably cancerous tissue. The organoids may be derived from colon cancer tissue, pancreatic cancer tissue, oesophageal cancer tissue, breast cancer tissue, lung cancer tissue, ovary cancer tissue, or prostate cancer tissue. Advantageously, the organoids may be derived from colon cancer tissue, pancreatic cancer tissue, or oesophageal cancer tissue. In accordance with any aspect, the organoids may be mammalian organoids, preferably from human or mouse.

Embodiments of the present invention will now be described by way of examples and not limited thereby, with reference to the accompanying figures. However, various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1. Exploration of techniques for large scale expansion of organoids. A. Representative images of COLO-005 6 days post seeding into ultra-low adherence 6 well plates in the detailed BME conditions. B. Representative images of COLO-005 6 days post seeding into conventional 6 well plates in the detailed BME conditions. C. Representative images of COLO-005 6 days post seeding into cell-repellent 6 well plates in the detailed BME conditions. D. Representative images of all colon lines tested, following seeding and growth for 3-7 days in the 80% BME domes vs. 5% BME suspension conditions. E. Representative images of all oesophageal lines tested, following seeding and growth for 3-7 days in the 80% BME domes vs. 5% BME suspension conditions. F. Representative images of all pancreatic lines tested, following seeding and growth for 3-7 days in the 80% BME domes vs. 5% BME suspension conditions. G. Top row—10× images of 3 organoid models growing in 5% BME. Bottom row—images of the same 3 organoid models growing in T75 flasks in 5% BME. H. Representative images of H&E staining and IHC of panCK (pan-cytokeratin), Ki67 and p53 in two organoids cultured in either 80% ECM domes or 5% ECM suspension. All images taken at 10× magnification.

FIG. 2. Longitudinal comparison study of 80% BME domes vs. 5% BME suspension conditions. A. Schematic of the longitudinal comparison study, each data point represents a passage of the model, the colours denote TO, T1 and T2 where organoids were harvested for whole genome sequencing (WGS) and RNA sequencing (RNAseq), and the box shows where drug screening was performed on the models. B. Representative images from 3 organoid lines over the first 7 weeks of the longitudinal experiment.

FIG. 3. Genomic stability analysis. Correlation density plot for 3 colon samples (top) and 3 oesophageal samples (bottom) in the 5% BME suspension cultures, showing the VAF for all (synonymous and non-synonymous) variants at TO (x axis) compared to all other time points (y axis).

FIG. 4. Genomic stability analysis. Correlation density plot for 3 colon samples (top) and 3 oesophageal samples (bottom) in the 5% BME suspension cultures, showing the VAF for non-synonymous variants at TO (x axis) compared to all other time points (y axis).

FIG. 5. Genomic stability analysis. Bar plot showing the average number of mutations per million bases, for each organoid line and time points, (grouped by organoid line, each group comprising a bar for, from left to right: TO, T1-80% BME, T1-5% BME, T2-80% BME, T2-5% BME).

FIG. 6. Genomic stability analysis. Circos plots showing variability across conditions and time points in terms of mutations and copy number, for 3 colon lines and 3 oesophageal lines. In each plot, the outer 3 tracks show the distribution of variants across the genome in a 5 mb window at TO and T2, and in the inner 3 tracks the log R copy number at TO and T2. Track 1 (outer)—T0 mutational spectrum, Track 2—5% ECM mutational spectrum, Track 3—80% ECM mutational spectrum, Track 4—T0 copy number, Track 5—5% ECM copy number, Track 6 (inner)—80% copy number.

FIG. 7. Genomic stability analysis. Bar plot showing the percentages of concordant (present in both the 80% BME and the 5% BME conditions) and discordant (present in either the 80% BME or the 5% BME conditions) mutations with a VAF of greater than 0.05. For each model (group of two bars), the left hand bar indicates T1, right hand bar T2. The colour of the bar represents the categories of the mutations: in each bar, the red part (bottom part) shows the percentage of concordant mutations, the blue (middle part) is the percentage of mutations exclusive to the 80% culture and green (top part) to the 5% culture. The right hand y axis shows the total number of variants considered in each bar. The squares below the barplot are coloured according to the value of the Jaccard index for all samples of the organoid line and time point above.

FIG. 8. Genomic stability analysis. Density plots for the Jaccard index when comparing within (tall peaks on the right side of each plot) and between different models (low peaks on the left side of each plot), VAF >=0.05. (A) colon samples, all variants on the top, non-synonymous on the bottom. (B) oesophageal samples, all variants on the top, non-synonymous on the bottom.

FIG. 9. Genomic stability analysis—Copy number driven cancer genes. Heatmap showing the copy number (log R, log 2(observed probe intensity/reference probe intensity)) of known copy number driven cancer genes in the organoids lines at TO and after culture in 5% BME or 80% BEM at T1 and T2, for both oesophageal (B) and colon cancer (A). C. Heatmap for all samples showing the log R copy number of all genes (0291-C15=OESO-009, 0309-C15=OESO-146, 0266-C20=COL-005, 0270-C20=COLO-021, 0300-C15=OESO-103, 0532-C20=COLO-133).

FIG. 10. Genomic stability analysis—cancer driver mutations. Intogen (https://www.intogen.org/) filtered driver variant heat maps for colon samples (left) and oesophageal samples (right), showing the VAF for each mutation.

FIG. 11. Genomic stability analysis—RNAseq analysis. A. Heatmap showing the correlation of the gene expression of 12,228 protein-coding genes (the colour bars show in the following order: top most: 0270, 0291, 0309, 0532, 0266 and 0300); second from top: colo, oeso, colo, oeso). B. Unsupervised hierarchical clustering of log transcript per million (TPM), for the 3,000 most differentially expressed genes (the colour bars show in the following order: top most: 0300, 0309, 0291, 0270, 0532, 0266; second from top: oeso, oeso, oeso, colo, colo, colo). The colour scale shows square root of standardised log 2 TPM values, scaled by the standard deviation and centred to the mean. *Some conditions were lost due to technical issues.

FIG. 12. Phenotypic assay results (drug activity screen) in standard (80% BME domes) vs low percentage matrix conditions. Correlation of activity plot of 4 organoid lines (top left: COLO-005, top right: COLO-021, bottom left: COLO-133, bottom right: OESO-009) treated with 72 drugs, 1-AUC in 5% culture condition shown on the x axis, and 1-AUC in 80% culture condition shown on the y axis.

FIG. 13. Phenotypic assay results (drug activity screen) in standard (80% BME domes) vs low percentage matrix conditions. A. Representative dose response curves for four organoid models (when treated with SCH772984. B. Representative dose response curves for four organoid models when treated with nutlin. Cells previously cultured in 5% ECM coloured green and 80% ECM coloured orange. All 3 biological and 3 technical replicates are shown, with the fitted dose response curve. C-F. Detailed data for COLO-005 (0266-C20, C), COLO-133 (0532-C20, D), COLO-021 (0270-C20, E), OESO-009 (0291-C15, F): activity (1-AUC) for each of the drugs tested, in the 5% BME condition (on the left of each subplot, as indicated on the x axis) and the 80% BME condition (as indicated on the x axis). Circles and triangles represent two separate technical replicates, each including biological triplicates. G. Dose response curves showing response of 4 organoid models to PF4708671 in both low ECM and standard conditions, dotted lines indicate the minimum and maximum drug concentration assayed. H. Activity plots for the 4 organoid models comparing response to the compound PF4708671 in low ECM and standard conditions.

FIG. 14. Phenotypic assay results (CRISPR screens) in standard (80% BME domes) vs low percentage matrix conditions. Schematic of the comparative CRISPR screens. Diagrams depict how the models were cultured throughout the screens

FIG. 15. Phenotypic assay results (CRISPR screens) in standard (80% BME domes) vs low percentage matrix conditions. ROC curves of the CRISPR screens performed in both culture conditions, for essential genes (A) and non-essential genes (B). The plots show a ROC curve for each of three biological replicates in the 5% and 80% conditions.

FIG. 16. Phenotypic assay results (CRISPR screens) in standard (80% BME domes) vs low percentage matrix conditions. Fold changes for genes (A) and sgRNA (B) for each of three biological replicates in the 5% and 80% conditions.

FIG. 17. Phenotypic assay results (CRISPR screens) in standard (80% BME domes) vs low percentage matrix conditions. Correlation plots showing the correlation of the log 2 fold changes at the gene level in the 5% (x axis) and 80% (y axis) COLO-027 screens (data combined across replicates in A and separately for each biological replicate in B).

FIG. 18. Phenotypic assay results (CRISPR screens) in standard (80% BME domes) vs low percentage matrix conditions. Plots showing all genes ranked by their log fold change in the 80% BME condition (top) and 5% BME condition (bottom), highlighted are two known model specific vulnerabilities also identified are the top 10% of all genes (horizontal line).

FIG. 19. Culture of organoids in very low ECM concentrations. A. Representative image of COLO-0167 organoids 7 days post seeding into ultra-low adherence 6 well plates in 2.5% BME. B. Representative image of COLO-0167 7 days post seeding into ultra-low adherence 6 well plates in 5% BME. C. Representative images of PANC-067 organoids 8 days post seeding into ultra-low adherence 6 well plates in 5% (2.5%) BME. D. Representative images of PANC-067 organoids 8 days post seeding into ultra-low adherence 6 well plates in 5% BME.

### DETAILED DESCRIPTION OF THE INVENTION

In describing the present invention, the following terms will be employed, and are intended to be defined as indicated below. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Organoid” as used herein in accordance with any aspect of the present invention may be a three-dimensional multicellular construct derived from primary tissue (e.g. from a subject) or pluripotent stem cells (such as embryonic stem cells (ESCs) or induced pluripotent stem cells (iPSCs)) which can self-organise and self-renew and replicate at least some of the organ functionalities of the tissue from which they are derived. Cells from which organoids can be derived will be referred to herein as “organoid progenitor cells”. These include cells from primary tissue and pluripotent stem cells.

Organoids may be obtained from various types of primary tissue, including cancerous and non-cancerous (also referred to as “untransformed”) tissue. Primary tissue refers to tissue that has been isolated from a subject. By extension, primary cells refer to cells that have been obtained from a primary tissue sample. Organoids may be derived from epithelial cells from primary tissue. Organoids have been derived from primary tissue from the colon (including healthy and colorectal carcinoma), pancreas (including healthy and pancreatic cancer), oesophagus (including healthy and oesophageal cancer), stomach (including healthy and stomach cancer), intestine (including healthy and bowel cancer), liver (including healthy and liver cancer), prostate (including healthy and prostate cancer), mammary gland (including healthy and breast cancer), biliary tree (also known as biliary tract, and including healthy and cancer biliary epithelium).

Organoids may also be obtained from pluripotent stem cells (PSCs), including induced pluripotent stem cells and embryonic stem cells. For example, brain, cardiac, kidney, stomach, liver, intestine, lung, and biliary tree organoids have been derived from PSCs.

In embodiments, the organoid(s) is/are colon, pancreas, oesophagus, prostate, breast, ovary or lung organoid(s). In particular, colon, pancreas, and oesophagus are described in detail herein. The organoids are preferably mammalian organoids, preferably from human or mouse. Human organoids are described in detail herein. Further, the organoids are preferably derived from primary tissue, including healthy and/or cancerous tissue. In embodiments, the organoid(s) is/are derived from cancerous cells. For example, organoids derived from colon cancer, pancreatic cancer, oesophageal cancer, ovarian cancer, lung cancer, breast cancer, and prostate cancer have been successfully cultured by the inventors according to the invention. In particular, organoids from colon cancer, pancreatic cancer and oesophageal cancer are described in detail herein. Without wishing to be bound by any particularly theory, the present inventors have demonstrated the invention with a variety of organoids derived from cancer tissue, and believe that the invention should be applicable to all cancer tissues from which organoids can be derived, and in particular to all cancer tissues from which organoids have been obtained using conventional culture protocols in domes of support matrix. Further, organoids have been derived from healthy/non-cancerous cells, for example using tissue surrounding tumours, and successfully cultured using conventional culture protocols in domes of support matrix. Such organoids should also be amenable to culture as described herein.

“Subject” as used herein in accordance with any aspect of the present invention is intended to be equivalent to “patient” and specifically includes both healthy individuals and individuals having a disease or disorder (e.g. a proliferative disorder such as a cancer). The subject may be a human, a companion animal (e.g. a dog or cat), a laboratory animal (e.g. a mouse, rat, rabbit, pig or non-human primate), an animal having a xenografted or xenotransplanted tumour or tumour tissue (e.g. from a human tumour), a domestic or farm animal (e.g. a pig, cow, horse or sheep). Preferably, the subject is a human subject.

The present disclosure provides compositions suitable for expansion of organoids. The compositions comprise a culture medium and a scaffold matrix, wherein the scaffold matrix is present in the composition at a concentration that is equivalent to between 2% (v/v) and 18% (v/v) of a complex protein hydrogel having a protein concentration between 12 and 18 mg/ml. The scaffold matrix is preferably present in the composition at a concentration that is equivalent to a concentration of at least 2.5% (v/v), at least 3% (v/v) or at least 4% (v/v) of a complex protein hydrogel having a protein concentration between 12 and 18 mg/ml.

As used herein, a “scaffold matrix” refers to a 3D scaffold matrix that supports the growth and proliferation of cells in 3-dimensions. Preferably, the scaffold matrix mimics the (natural) extracellular matrix by its interaction with cellular membrane proteins such as integrins. Without wishing to be bound by any particular theory, the present inventors believe that any scaffold matrix that has been used for culture of organoids using conventional protocols in which the organoids are embedded in a dome of scaffold matrix would be suitable for use according to the present invention. Further, for a particular matrix (even a matrix that is not a complex protein hydrogel), the concentration that is equivalent to between 2% (v/v) and 18% (v/v) of a complex protein hydrogel having a protein concentration between 12 and 18 mg/ml (hereafter “reference matrix”) may be determined by comparing the concentration of the reference matrix when used in a conventional dome-based culture process with the equivalent concentration used for the alternative matrix in a conventional dome-based culture process. For example, a product such as Cultrex™ BME (which is a “reference matrix” as described above) is typically used in a conventional dome-based culture process at a concentration of approx. 80% (v/v) to 100% (v/v). Such a reference matrix has been demonstrated by the inventors to be usable within the context of the present invention at concentrations between 2 and 18% (v/v), i.e. between 0.02 (2%/100%) and 0.225 (18%/80%) times the concentration used in a conventional dome-based culture process. Therefore, another matrix may suitably be used at a concentration that is between about 0.02 and about 0.225 of the concentration at which the matrix is used to culture organoids embedded in domes of matrix.

As the skilled person understands, a scaffold matrix may comprise a single material or a plurality of materials. For example, synthetic matrices supplemented with BME have been previously used. The concentrations described herein refer to the total concentration (or equivalent concentration) of matrix material in the composition. For example, a composition comprising 3 mg/ml fibrinogen and 10% (v/v) matrigel polymerises to form a continuous solid matrix due to the presence of the fibrinogen and is hence functionally equivalent to a composition comprising 80-100% (v/v) matrigel. A composition according to the present disclosure could be formulated based on such a composition, for example by including between 0.06 mg/ml fibrinogen and 0.2% (v/v) matrigel (0.02 times the concentrations used to form a continuous matrix) and 0.675 mg/ml fibrinogen and 2.25% (v/v) matrigel (0.225 times the concentrations used to form a continuous matrix).

The scaffold matrix is preferably a hydrogel. For example, a scaffold matrix may be a complex protein hydrogel (such as basement membrane extract) typically obtained from tissues or cells, or a synthetic polymer hydrogel (such as polyglycolic acid (PGA) hydrogels and crosslinked dextran and PVA hydrogels (e.g. Cellendes Gmbh, Reutlingen Del.)). Preferably, the scaffold matrix is a complex protein hydrogel. Complex protein hydrogels may comprise extracellular matrix components, such as laminin, collagen IV, enactin and heparin sulphate proteoglycans. Complex protein hydrogels may also include hydrogels of extracellular matrix proteins from Engelbreth-Holm-Swarm (EHS) mouse sarcoma cells. Suitable complex protein hydrogels are available from commercial sources and include Matrigel™ (Corning Life Sciences) or Cultrex™ BME 2. Preferably, the scaffold matrix is a laminin-containing extracellular matrix such as a basement membrane or basement membrane extract. Preferably, the scaffold matrix is a basement membrane extract, such as a soluble form of basement membrane purified from Engelbreth-Holm-Swarm (EHS) tumour cells. A particularly suitable basement membrane extract is Cultrex™ Basement Membrane Extract, preferably Cultrex™ BME, Cultrex™ BME type 3 or Cultrex™ BME type 2 (also referred to herein as “BME2”). BMEs (such as Cultrex™ BME, Cultrex™ BME type 2, Cultrex™ BME type 3, and Corning™ Matrigel™) are typically provided as complex compositions of extracellular matrix components in a buffer. For example, Cultrex™ BME, Cultrex™ BME type 3 and Cultrex™ BME type 2 use DMEM as a buffer. Due to their complex composition which has inherent variability (as it is purified from cell cultures), they are frequently characterised in terms of their range of total protein concentration per ml. A typical BME is provided as a solution with a total protein concentration between 8 and 22 mg/ml. Cultrex™ BME, Cultrex™ BME type 3 and Cultrex™ BME type 2 are provided as solutions with a total protein concentration between 12 and 18 mg/ml. Corning™ Matrigel™ is provided in a standard formulation with a protein concentration between 8 and 12 mg/ml, and in a high protein formulation with a protein concentration between 18 and 22 mg/ml.

All complex protein hydrogel concentrations provided herein are provided by reference to BMEs having a protein concentration between 12 and 18 mg/ml, unless indicated otherwise. For example, when the scaffold matrix is a complex protein hydrogel having a protein concentration between 12 and 18 mg/ml, this scaffold matrix may be present in the composition at a concentration between 2% (v/v) and 18% (v/v). As a particular example, such a composition may comprise a culture medium and Cultrex™ BME, Cultrex™ BME type 3 or Cultrex™ BME type 2 at a concentration between 2% (v/v) and 18% (v/v). In such a composition, the protein concentration from the complex protein hydrogel may be between 0.24 mg/ml and 3.24 mg/ml. As such, the compositions described herein may equally be described as compositions wherein the scaffold matrix is present in the composition at a concentration that is equivalent to a concentration of complex protein hydrogel resulting in between 0.24 mg/ml and 3.24 mg/ml protein from the complex protein hydrogel. As another example, such a composition may comprise a culture medium and Cultrex™ BME, Cultrex™ BME type 3 or Cultrex™ BME type 2 at a concentration of approximately 5% (v/v). In such a composition, the protein concentration from the complex protein hydrogel may be between 0.6 mg/ml (5%*12 mg/ml) and 0.9 mg/ml (5%*18 mg/ml). Therefore, such a particularly advantageous composition may equally be described as a composition wherein the scaffold matrix is present in the composition at a concentration that is equivalent to a concentration of complex protein hydrogel resulting in between 0.6 mg/ml and 0.9 mg/ml protein from the complex protein hydrogel.

In embodiments, the scaffold matrix is present in the composition at a concentration that is equivalent to a concentration of between 2.5% (v/v) and 18% (v/v), between 2.5% (v/v) and 15% (v/v) between 3% (v/v) and 18% (v/v), between 3% (v/v) and 15% (v/v), between 4% (v/v) and 15% (v/v), between 4% (v/v) and 12% (v/v), between 4% (v/v) and 10% (v/v), or between 5% (v/v) and 10% (v/v), of a complex protein hydrogel having a protein concentration between 12 and 18 mg/ml. As explained above, the scaffold matrix may therefore be present in the composition at a concentration that is between 0.025 and 0.225, between 0.025 and 0.1875, between 0.03 and 0.225, between 0.03 and 0.1875, between 0.04 and 0.1875, between 0.04 and 0.15, between 0.04 and 0.125, between 0.05 and 0.125 times the concentration of the scaffold matrix that is usable to culture organoids embedded in domes of the scaffold matrix (i.e. according to a conventional dome-based culture protocol). For example, when the scaffold matrix is a complex protein hydrogel having a protein concentration between 12 and 18 mg/ml, this scaffold matrix may be present in the composition at a concentration between 2% (v/v) and 18% (v/v), between 2.5% (v/v) and 18% (v/v), between 3% (v/v) and 18% (v/v), between 4% (v/v) and 18% (v/v), between 3% (v/v) and 15% (v/v), between 4% (v/v) and 15% (v/v), between 4% (v/v) and 12% (v/v), between 4% (v/v) and 10% (v/v), or between 5% (v/v) and 10% (v/v). In embodiments, the scaffold matrix is present in the composition at a concentration that is equivalent to a concentration of at least 2% (v/v), at least 2.5% (v/v), at least 3% (v/v), at least 4% (v/v), or at least 5% (v/v), of a complex protein hydrogel having a protein concentration between 12 and 18 mg/ml. As such, the scaffold matrix may be present in the composition at a concentration that is at least 0.02, at least 0.025, at least 0.03, at least 0.04, or at least 0.05 times the concentration of the scaffold matrix that is usable to culture organoids embedded in domes of the scaffold matrix. In such embodiments, the scaffold matrix may be present in the composition at a concentration that is equivalent to a concentration of at most at most 18% (v/v), at most 17% (v/v), at most 16% (v/v), at most 15% (v/v), at most 14% (v/v), at most 13% (v/v), at most 12% (v/v), at most 11% (v/v), or at most 10% (v/v), of a complex protein hydrogel having a protein concentration between 12 and 18 mg/ml. As such, the scaffold matrix may be present in the composition at a concentration that is at most 0.225, at most 0.2125, at most 0.2, at most 0.1875, at most 0.175, at most 0.1625, at most 0.15, at most 0.1375, or at most 0.125 times the concentration of the scaffold matrix that is usable to culture organoids embedded in domes of the scaffold matrix.

For example, an ECM matrix derived from decellularised porcine intestine was shown to be usable for culture of organoids in domes of ECM gels at a concentration of 4-6 mg/ml in Giobbe et al. (2019). Such a scaffold matrix could be used according to the invention at a concentration of at least 0.08 mg/ml (0.02 times 4 mg/ml), at least 0.10 mg/ml (0.025 times 4 mg/ml), at least 0.12 mg/ml (0.03 times 4 mg/ml), at least 0.16 mg/ml (0.04 times 4 mg/ml), or at least 0.20 (0.05 times 4 mg/ml), and at most 1.35 mg/ml (0.225 times 6 mg/ml), at most 1.275 mg/ml (0.2125 times 6 mg/ml), at most 1.2 mg/ml (0.2 times 6 mg/ml), at most 1.125 mg/ml (0.1875 times 6 mg/ml), at most 1.0 mg/ml (0.175 times 6 mg/ml), at most 0.975 mg/ml (0.1625 times 6 mg/ml), at most 0.9 mg/ml (0.15 times 6 mg/ml), at most 0.825 mg/ml (0.1375 times 6 mg/ml), or at most 0.75 mg/ml (0.125 times 6 mg/ml).

Engineered materials suitable for use as scaffold matrices in conventional (dome-based) organoid culture protocols were reviewed in Kratochvil et al. (2019). Any of those materials may be used in the content of the present disclosure, in concentrations calculated as explained above. For example, PEG (polyethylene glycol, e.g. transglutaminase (TG) cross-linked PEG), functionalised PEG (e.g. PEG modified with a fibronectin derived RGD peptide, laminin-derived peptides, fibronectin-derived peptides containing both the RGD motif and the PHSRN synergy site, or collagen I-derived peptide), PEG-alginate matrices have been used to culture organoids embedded in matrix (see e.g. Gjorevski, N., Sachs, N., Manfrin, A. et al. 2016; Hernandez-Gordillo et al., 2019; Broguiere et al., 2018). Any of those materials may be used in the content of the present disclosure, in concentrations calculated as explained above.

As another example, hyaluronan (HA) gels (e.g. semisynthetic TG cross-linked hyaluronan (HA) gel), alginate gels (e.g. natural calcium cross-linked alginate gel), and fibrin gels (e.g. human-derived thrombin cross-linked fibrin gel) have been used with or without BME supplementation (see e.g. Broguiere et al., 2018). Any of those materials may be used in the content of the present disclosure, in concentrations calculated as explained above. In convenient embodiments, the scaffold matrix is present in the composition at a concentration that is equivalent to a concentration of about 5% (v/v) of a complex protein hydrogel having a protein concentration between 12 and 18 mg/ml. For example, when the scaffold matrix is a complex protein hydrogel having a protein concentration between 12 and 18 mg/ml, this scaffold matrix may be present in the composition at a concentration of about 5% (v/v). As explained above, the scaffold matrix may be present at a concentration of about 0.05-0.0625 (5%/80% to 5%/100%) of the concentration that is used to culture organoids according to a conventional dome-based protocol, using the chosen scaffold matrix.

As the skilled person understands, equivalent concentrations using different complex protein hydrogels can also be determined by comparing the protein concentration of such complex protein hydrogels with that of a reference protein hydrogel having a protein concentration between 12 and 18 mg/ml. For example, when using a scaffold matrix that is a complex protein hydrogel (e.g. a BME) having a protein concentration between 8 and 12 mg/ml (such as e.g. Corning™ Matrigel™ standard formulation), the scaffold matrix may be present in the composition at a concentration between 2% (v/v) and 40.5% (v/v) (or between 2.5% (v/v) and 40.5% (v/v), between 3% (v/v) and 40.5% (v/v), between 4% (v/v) and 40.5% (v/v), between 3% (v/v) and 33.75% (v/v), between 4% (v/v) and 33.75% (v/v), between 4% (v/v) and 27% (v/v), between 4% (v/v) and 22.5% (v/v), or between 5% (v/v) and 22.5% (v/v)). Similarly, when using a scaffold matrix that is a complex protein hydrogel (e.g. a BME) having a protein concentration between 18 and 22 mg/ml (such as e.g. Corning™ Matrigel™ high protein formulation), the scaffold matrix may be present in the composition at a concentration between 1.1% (v/v) and 18% (v/v). In specific examples, when using a scaffold matrix that is a complex protein hydrogel (e.g. a BME) having a protein concentration between 18 and 22 mg/ml (such as e.g. Corning™ Matrigel™ high protein formulation), the scaffold matrix may be present in the composition at a concentration between 1.35% (v/v) and 18% (v/v), between 1.35% (v/v) and 15% (v/v), between 1.6% (v/v) and 18% (v/v), between 1.6% (v/v) and 15% (v/v), between 2.18% (v/v) and 18% (v/v), between 2.18% (v/v) and 15% (v/v), between 2.18% (v/v) and 12% (v/v), between 2.18% (v/v) and 10% (v/v), or between 2.73% (v/v) and 10% (v/v).

In preferred embodiments, the compositions comprise a culture medium and a scaffold matrix that is a complex protein hydrogel (such as e.g. a BME), wherein the complex protein hydrogel is present in the composition at a concentration that is equivalent to between 2% (v/v) and 18% (v/v) of a complex protein hydrogel having a protein concentration between 12 and 18 mg/ml. In embodiments, the complex protein hydrogel is present in the composition at a concentration that results in a protein concentration from the complex protein hydrogel of between 0.24 mg/ml and 3.24 mg/ml. In particular embodiments, the complex protein hydrogel is present in the composition at a concentration that results in a protein concentration from the complex protein hydrogel of between 0.24 mg/ml and 3.24 mg/ml, between 0.30 mg/ml and 3.24 mg/ml, between 0.36 mg/ml and 3.24 mg/ml, between 0.48 mg/ml and 3.24 mg/ml, between 0.48 mg/ml and 2.7 mg/ml, between 0.48 mg/ml and 2.16 mg/ml, between 0.48 mg/ml and 1.8 mg/ml, or between 0.6 mg/ml and 1.8 mg/ml. In particular embodiments, the complex protein hydrogel is present in the composition at a concentration that results in a protein concentration from the complex protein hydrogel of at least 0.24 mg/ml, at least 0.30 mg/ml, at least 0.36 mg/ml, at least 0.48 mg/ml, or at least 0.6 mg/ml. In such embodiments, the complex protein hydrogel may be present in the composition at a concentration that results in a protein concentration from the complex protein hydrogel of at most 3.24 mg/ml, at most 3.06 mg/ml, at most 2.88 mg/ml, at most 2.7 mg/ml, at most 2.52 mg/ml, at most 2.34 mg/ml, at most 2.16 mg/ml, at most 1.98 mg/ml, or at most 1.8 mg/ml.

A “culture medium” refers to a composition that comprises at least nutrients and additional factors that support the proliferation of organoids (such as e.g. growth factors, mitogens and pathway modulators). Within the context of the present invention, a culture medium is typically a liquid composition. A culture medium may be a chemically defined medium. A chemically defined medium is a nutritive solution for culturing cells which contains only specified components, preferably components of known chemical structure. A chemically defined medium is devoid of undefined components or constituents which include undefined components, such as feeder cells, stromal cells, serum, serum albumin and complex extracellular matrices, such as Matrigel™. A chemically defined medium may be humanised. A humanised chemically defined medium is devoid of components or supplements derived or isolated from non-human animals, such as Foetal Bovine Serum (FBS) and Bovine Serum Albumin (BSA), and mouse feeder cells. Conditioned medium includes undefined components from cultured cells and is not chemically defined. A “concentrated culture medium” (or “concentrated medium”) refers to a composition that is designed to be diluted prior to being used as a culture medium, for example using water (distilled or sterilised, as appropriate) or a buffer. In other words, a concentrated version of a culture medium is a composition that contains the ingredients of a culture medium in concentrations higher than those intended for use as a culture medium.

A culture medium typically comprises a basal medium. Suitable basal media include Iscove's Modified Dulbecco's Medium (IMDM), Ham's F12, Advanced Dulbecco's modified eagle medium (DMEM) or DMEM/F12 (Price et al Focus (2003), 25 3-6), Williams E (Williams, G. M. et al Exp. Cell Research, 89, 139-142 (1974)), and RPMI-1640 (Moore, G. E. and Woods L. K., (1976) Tissue Culture Association Manual. 3, 503-508. In embodiments, advanced DMEM is preferred.

The basal medium may be supplemented with a media supplement, such as N2 (Gibco), B-27™ (ThermoFisher) and/or one or more additional supplements which may include L-glutamine or substitutes, such as L-alanyl-L-20 glutamine (e.g. Glutamax™), nicotinamide, N-acetylcysteine, buffers, such as HEPES, and antibiotics such as blasticidin or puromycin. B-27™ is a serum-free supplement that is commonly used for neural cell cultures. N2 is a chemically-defined, serum-free supplement based on Bottenstein's N-1 formulation. It is commonly used for neuroblastoma and neuron cultures. For example, the basal medium (e.g. advanced DMEM) may be supplemented with HEPES, Glutamax, N2 and optionally nicotinamide and/or N-acetylcysteine. Such a medium may be a chemically defined medium.

Organoid models typically require a tailored culture medium formulation including additional factors that support their proliferation and/or differentiation. These may include pathway modulators, vitamins and tropic mitogens (reviewed in Baker et al., (2016) and Merker et al. (2016)). For example, the culture medium may additionally comprise one or more compounds selected from: growth factors (such as epidermal growth factor (EGF), fibroblast growth factor 10 (FGF10)), a TGFβ inhibitor, a non-canonical Wnt signalling potentiator, a BMP inhibitor, hormones (such as e.g. gastrin and/or prostaglandin E2), a canonical Wnt ligand, and a p38 MAPK signalling inhibitor.

Epidermal Growth Factor (EGF; NCBI GeneID: 1950, nucleic acid sequence NM_001178130.1 GI:296011012; amino acid sequence NP_001171601.1 GI: 296011013) is a protein factor which stimulates cellular growth, proliferation and cellular differentiation by binding to an epidermal growth factor receptor (EGFR). EGF may be produced using routine recombinant techniques or obtained from commercial suppliers (e.g. R&D Systems, Minneapolis, Minn.; Stemgent Inc, USA). Suitable concentrations of EGF for expanding organoids are known in the art and may be readily determined using standard techniques.

A TGFβ inhibitor is a compound that reduces, blocks or inhibits TGFβ signalling through the TGFβRI and 15 TGFβRII receptors. Suitable TGFβ inhibitors include A83-01 3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), GW788388 (4-[4-[3-(2-Pyridinyl)-1H-pyrazol-4-yl]-2-pyridinyl]-N-(tetrahydro-2H-pyran-4-yl)-benzamide), IN1130 (3-[[5-(6-Methyl-2-pyridinyl)-4-(6-quinoxalinyl)-1H-imidazol-2-yl]methyl]benzamide), LY364947 (4-[3-(2-Pyridinyl)-1H-pyrazol-4-yl]-quinoline), SB525334 (6-[2-(1,1-Dimethylethyl)-5-(6-methyl-2-pyridinyl)-1H-imidazol-4-yl]quinoxaline), SB431542 (4-(5-Benzol[1,3]dioxol-5-yl-4-pyridin-2-yl-1H-imidazol-2-yl)-benzamide hydrate; Sigma, Tocris Bioscience, Bristol UK), SB-505124 (2-(5-benzo[1,3]dioxol-5-yl-2-tert-butyl-3H-imidazol-4-yl)-6-methylpyridine hydrochloride) and soluble protein factors, such as lefty (e.g. human lefty 2: NP_003231.2 GI:27436881), cerberus (e.g. human Cerberus 1: NP_005445.1 GI:4885135) and follistatin (e.g. human foistatin: NP_006341.1 GI:5453652). Suitable TGFβ inhibitors are available from commercial suppliers. In some embodiments, the TGFβ inhibitor may be A8301 (also referred to herein as A83-01).

A BMP inhibitor is a compound that reduces, blocks or inhibits the activity of bone morphogenetic protein (BMP) ligands of the transforming growth factor beta (TGF-β) family. A BMP inhibitor may be a BMP antagonist, and may bind to and antagonise one or more BMPs. Suitable BMP antagonists include Noggin. Noggin inhibits a least BMP2, BMP4, BMP5, BMP6, BMP7, BMP13, and BMP14. Noggin is a secreted polypeptide that diffuses through extracellular matrices more efficiently than members of the TGF-β family. Preferably, noggin is human noggin, encoded by the gene NOG (GeneID 9241, which encodes the noggin precursor with nucleic acid sequence reference NM_005450.6 and amino acid sequence reference NP_005441.1). Noggin is readily available from commercial sources (e.g. ThermoFisher).

Suitable concentrations of noggin for expanding organoids are known in the art and may be readily determined using standard techniques.

A non-canonical Wnt signalling potentiator is a compound that stimulates, promotes or increases the activity of the non-canonical Wnt signalling pathway. A non-canonical Wnt signalling potentiator may selectively potentiate non-canonical Wnt signalling or more preferably, may potentiate both the non-canonical Wnt signalling and the canonical Wnt signalling pathway (i.e. a Wnt signalling agonist). Preferred non-canonical Wnt signalling potentiators include the Wnt signalling agonist R-spondin. R-spondin is a secreted activator protein with two cysteine-rich, furin-like domains and one thrombospondin type 1 domain that positively regulates Wnt signalling pathways. Preferably, R-spondin is human R-spondin. R-spondin may include RSPO1 (GeneID 284654 nucleic acid sequence reference NM_001038633.3, amino acid sequence reference NP_001033722.1), RSPO2 (GeneID 340419 nucleic acid sequence reference NM_001282863.1, amino acid sequence reference NP_001269792.1), RSPO3 (GeneID 84870, nucleic acid sequence reference NM_032784.4, amino acid sequence reference NP_116173.2) or RSPO4 (GeneID 343637, nucleic acid sequence reference NM_001029871.3, amino acid sequence reference NP_001025042.2). R-spondin is readily available from commercial sources (e.g. R&D Systems, Minneapolis, Minn.). Suitable concentrations of R-spondin for expanding organoids are known in the art and may be readily determined using standard techniques.

A canonical Wnt ligand is a secreted lipid-modified glycoprotein that activates the Wnt signalling pathway by binding to a Frizzled (Fz) family receptor. Wnt ligands include, in human, WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16. A preferred canonical Wnt ligand is WNT3A. Preferably, WNT3A is human WNT3A, encoded by the gene WNT3A (Gene ID 89780, which encodes the precursor having nucleic acid sequence NM_033131.4 and amino acid sequence NP_149122.1). WNT3A is readily available from commercial sources (e.g. Sigma-Aldrich). Suitable concentrations of WNT3A for expanding organoids are known in the art and may be readily determined using standard techniques.

Gastrin is a peptide hormone released by G cells in the pyloric antrum of the stomach, duodenum, and the pancreas. Gastrin acts as a mitogenic factor for gastrointestinal epithelial cells. Gastrin has two biologically active peptide forms, G34 and G17. Preferably, gastrin is human gastrin, encoded by the gene GAST (Gene ID 2520, which encodes the preproprotein having nucleic acid sequence NM_000805.5 and amino acid sequence NP_000796.1, from which the active peptide forms are derived). Gastrin is readily available from commercial sources (e.g. R&D Systems). Suitable concentrations of Gastrin for expanding organoids are known in the art and may be readily determined using standard techniques.

Prostaglandin E2 (PGE2), also known as dinoprostone, is a naturally occurring prostaglandin. Prostaglandins are physiologically active lipids of the eicosanoid category. PGE2 is also known as (Z)-7-[(1R,2R,3R)-3-hydroxy-2-[(E,3S)-3-hydroxyoct-1-enyl]-5-oxocyclopentyl]hept-5-enoic acid and 13E-dien-1-oic acid. PGE2 is a potent activator of the Wnt signaling pathway. It has been implicated in regulating the developmental specification and regeneration of hematopoietic stem cells through cAMP/PKA activity. PGE2 is readily available from commercial sources (e.g. R&D Systems). Suitable concentrations of PGE2 for expanding organoids are known in the art and may be readily determined using standard techniques.

Fibroblast growth factor 10 (FGF10; NCBI GeneID: 2255, nucleic acid sequence NM_004465.2; amino acid sequence NP_004456.1) is a protein factor which stimulates cellular proliferation and survival, and is involved in embryonic epidermal morphogenesis. FGF10 may be produced using routine recombinant techniques or obtained from commercial suppliers (e.g. R&D Systems, Minneapolis, Minn.; Stemgent Inc, USA). Suitable concentrations of FGF10 for expanding organoids are known in the art and may be readily determined using standard techniques.

A p38 MAPK signalling inhibitor is a compound that reduces, blocks or inhibits p38 MAPK signalling, preferably by inhibiting one or more p38 MAPKs. p38 MAPKs (p38-α (MAPK14), p38-β (MAPK11), p38-γ (MAPK12/ERK6), and p38-δ (MAPK13/SAPK4)) are members of the MAPK family that are activated by a variety of environmental stresses and inflammatory cytokines. As with other MAPK cascades, a membrane-proximal component referred to as a MAPKKK (MAP kinase kinase kinase) phosphorylates and activates MKK3/6 (MAP kinase kinase), the p38 MAPK kinases. These in turn phosphorylate and activate the p38 MAPK. MKK3/6 can also be activated directly by ASK1, which is stimulated by apoptotic stimuli. Suitable p38 MAPK inhibitors include SB202190 (4-(4-Fluorophenyl)-2-(4-hydroxyphenyl)-5-(4-pyridyl)-1H-imidazole), SB203580 (4-(4-Fluorophenyl)-2-(4-methylsulfinylphenyl)-5-(4-pyridyl)-1H-imidazole), doramapimod (1-[5-tert-butyl-2-(4-methylphenyl)pyrazol-3-yl]-3-[4-(2-morpholin-4-ylethoxy)naphthalen-1-yl]urea), ralimetinib (5-[2-tert-butyl-4-(4-fluorophenyl)-1H-imidazol-5-yl]-3-(2,2-dimethylpropyl)imidazo[4,5-b]pyridin-2-amine), VX-702 (6-(N-carbamoyl-2,6-difluoroanilino)-2-(2,4-difluorophenyl)pyridine-3-carboxamide), PD169316 (4-[4-(4-fluorophenyl)-2-(4-nitrophenyl)-1H-imidazol-5-yl]pyridine), TA-02 (4-[2-(2-fluorophenyl)-4-(4-fluorophenyl)-1H-imidazol-5-yl]pyridine), SD0006 (1-[4-[3-(4-chlorophenyl)-4-pyrimidin-4-yl-1H-pyrazol-5-yl]piperidin-1-yl]-2-hydroxyethanone), PH-797804 (3-[3-bromo-4-[(2,4-difluorophenyl)methoxy]-6-methyl-2-oxopyridin-1-yl]-N,4-dimethylbenzamide), VX-745 (5-(2,6-dichlorophenyl)-2-(2,4-difluorophenyl)sulfanylpyrimido[1,6-b]pyridazin-6-one), TAK-715 (N-[4-[2-ethyl-4-(3-methylphenyl)-1,3-thiazol-5-yl]pyridin-2-yl]benzamide), SB239063 (4-[4-(4-fluorophenyl)-5-(2-methoxypyrimidin-4-yl)imidazol-1-yl]cyclohexan-1-ol), skepinone-L (13-(2,4-difluoroanilino)-5-[(2R)-2,3-dihydroxypropoxy]tricyclo[9.4.0.03,8]pentadeca-1(11),3(8),4,6,12,14-hexaen-2-one), losmapimod (6-[5-(cyclopropylcarbamoyl)-3-fluoro-2-methylphenyl]-N-(2,2-dimethylpropyl)pyridine-3-carboxamide), praeruptorin A ([(9S,10S)-10-acetyloxy-8,8-dimethyl-2-oxo-9,10-dihydropyrano[2,3-f]chromen-9-yl] (Z)-2-methylbut-2-enoate), BMS-582949 (4-[5-(cyclopropylcarbamoyl)-2-methylanilino]-5-methyl-N-propylpyrrolo[2,1-f][1,2,4]triazine-6-carboxamide), pexmetinib (1-[5-tert-butyl-2-(4-methylphenyl)pyrazol-3-yl]-3-[[5-fluoro-2-[1-(2-hydroxyethyl)indazol-5-yl]oxyphenyl]methyl]urea), and UM-164 (2-[[6-[4-(2-hydroxyethyl)piperazin-1-yl]-2-methylpyrimidin-4-yl]amino]-N-[2-methyl-5-[[3-(trifluoromethyl)benzoyl]amino]phenyl]-1,3-thiazole-5-carboxamide). Suitable p38 MAPK inhibitors are available from commercial suppliers. In some embodiments, the p38 MAPK inhibitor may be SB202190 (which is available e.g. from Sigma Aldrich). Suitable concentrations of p38 MAPK inhibitor for expanding organoids are known in the art and may be readily determined using standard techniques.

In embodiments, a culture medium may comprise one or more of: Noggin, N-acetyl cysteine, Nicotinamide, EGF, Gastrin, A83-01, SB202190, Prostaglandin E2, R-spondin, WNT3A, B27, and FGF10. For example, for colon organoids (including colon cancer organoids), a culture medium may comprise: Noggin, N-acetyl cysteine, Nicotinamide, EGF, Gastrin, A83-01, SB202190, B27, and one or both of Prostaglandin E2, and R-spondin. For pancreas organoids (including pancreatic cancer organoids), a culture medium may comprise: Noggin, N-acetyl cysteine, Nicotinamide, EGF, A83-01, R-spondin, WNT3A, B27, and FGF10, and one or both of Gastrin and SB202190. For oesophagus organoids (including oesophageal cancer organoids), a culture medium may comprise: Noggin, N-acetyl cysteine, EGF, A83-01, SB202190, R-spondin, WNT3A, B27, and FGF10, and one or both of EGF and Gastrin. These compositions are provided merely as examples of compositions that may be used. Suitable culture medium compositions for expanding many different types of organoids have been proposed, and suitable compositions for any specific type of organoids may be determined using standard techniques.

The present disclosure also provides a method for producing an expanded population of organoids in vitro comprising:
(i) providing a population of organoid progenitor cells or organoids; and
(ii) culturing the population in a composition comprising a culture medium and a scaffold matrix as described herein, thereby producing an expanded population of organoids. The method may comprise preparing an organoid culture by mixing organoids or organoid progenitor cells, and a composition as described herein.

Step (ii) may comprise culturing the population in a composition comprising a culture medium and a scaffold matrix as described herein for a first period of time, and culturing the population in a composition comprising a culture medium and a scaffold matrix in a concentration that is at least half of that of the concentrations described herein for a further period of time. For example, this may be achieved by adding culture medium to the culture after the first period of time, in an amount up to 100% of the volume of the composition. The first period of time is preferably sufficient for the organoids or organoid progenitor cells to attach themselves to the particles of scaffold matrix in the composition. The predetermined period of time is preferably at least 24 hours, at least 48 hours, at least 72 hours, at least 96 hours, or at least a week.

Also described is a method of preparing an organoid culture, comprising mixing: organoids or organoid progenitor cells, and a composition as described herein, thereby forming said culture.

The organoid cultures described herein are preferably maintained in low adherence cell culture containers (also referred to herein as “low adhesion” or “low attachment” (LA), “ultra-low adherence”, “ultra-low adhesion”, or “ultra-low attachment” (ULA) or “cell repellent” (CR) culture containers). Indeed, the present inventors have found that organoids cultured in compositions as described herein had a tendency to adhere to the surface of cell culture containers if said containers were not low adhesion containers.

Cell culture containers (also referred to herein as “culture containers”) refer to recipients (including dishes, plates, flasks and tubes) that are suitable for or specifically designed to contain cell cultures. Culture containers can be reusable or disposable. Disposable cell culture containers are typically preferred to reduce the risk of contamination. As such, culture containers are typically made of plastic, preferably optically transparent plastic, such as polystyrene or polycarbonate. Glass cell culture containers can also be used. Preferred cell culture containers are made of polystyrene. Cell culture plates refer to multi-well containers, typically rectangular structures comprising a plurality (such as e.g. 4, 6, 8, 12, 24, 48, 96, 384 or 1536) of shallow (often round) wells. Cell culture dishes refer to single well shallow containers such as e.g. Petri dishes (which are circular shallow dishes, typically lidded) and rectangular cell culture dishes. Cell culture tubes refer to cylindrical or conical containers which can have a round or flat bottom surface, and are typically lidded. Cell culture flasks refer to containers that have a wider vessel “body” and one (or sometimes more) narrower tubular sections called necks that connect the body to an opening (which is typically lidded). Commonly used cell culture flasks have a parallelepiped shape with a flat (typically rectangular) bottom surface.

Cell culture containers may be uncoated and/or untreated or coated and/or treated on at least a part of their internal surface (typically either the whole of their internal surface or at least the part of their internal surface that will form the bottom surface of the container in use). Coatings can be applied without covalent bonds (also referred to as passive coatings) or can be bound covalently with the surface on which it is applied. Non-covalent coatings commonly used for cell culture containers include phospholipids, streptavidin, antibodies, collagen I and Poly-d-lysine (PDL). These can be used alone or in combinations. Covalent coatings commonly used for cell culture containers include some streptavidin coatings, nickel chelate, protein A, WGA (wheat germ agglutinin) and hydrogels. Surfaces of containers can be treated for example using energy based methods (e.g. plasma treatment), to change the physico-chemical properties of the surface. For example, the surface of a container can be treated to increase its hydrophilic character. This may help to improve cell adhesion as materials such as polystyrene are typically hydrophobic when untreated. Examples of such surface treatments include the Nunclon™ Delta surface treatment from Thermo Scientific™ and Corning™ TC-treated labware. For adherent cell cultures, coated and/or treated cell culture containers are typically preferred, where the coating/treatment is chosen to promote adhesion of cells to the coated surface. For example, collagen and or Poly-d-lysine (PDL) coated containers are frequently used. For suspension cultures, uncoated cell culture containers, or coated cell culture containers with anti-adhesion coatings are typically preferred.

Conventional or standard cell culture containers refer to cell culture containers that are treated to increase the hydrophilic character of the surface (e.g. Nunclon Delta treated containers, plasma treated containers), and/or coated to increase cell adhesion (typically with collagen I and poly-D-lysine). Low adherence cell culture containers refer to cell culture containers that are either untreated (where the material is naturally hydrophobic) or treated/coated to reduce cell adhesion. Examples of low adherence cell culture containers include those coated with a covalently bound hydrogel layer (such as Corning™'s ultra-low attachment range, Thermo Fisher™'s Nunclon™ Sphera™ range, or polyHEMA (poly-2-hydroxyethyl methacrylate) coated plates) or a covalently bound hydrophobic polymer (e.g. a hydrophobic fluorinated polymer), such as e.g. Greiner Bio-One's CellStar™ range.

The organoid cultures described herein are typically suspension cultures. The term “suspension culture” refers to the culture of a cell or organoid in a solution, where the cell or organoid does not adhere or attach to the surface of the container in which the culture is maintained, and is not supported by a fixed scaffold. By contrast, in an adherent culture the cells or organoids are supported on and adhered to the surface of the container in which the culture is maintained. In a 3D scaffold culture, the cells or organoids are embedded in (and supported by) a continuous scaffold which itself rests or is otherwise supported on the surface of the container in which the culture is maintained. In both 3D scaffold culture and adherent culture, a liquid culture medium is typically provided which surrounds the scaffold and embedded cells, or the adhered cells, respectively.

Organoid populations are typically cultured in 3D scaffold culture. In particular, a concentrated scaffold matrix solution comprising the organoid progenitor cells or organoids and a scaffold matrix is typically deposited in the forms of droplets (e.g. 10-50 μl) on a surface (e.g. a dish or well of a plate). The surface is typically coated priori to deposition of the droplets. For example, the surface may be pre-coated with a layer of the same scaffold matrix, which is optionally allowed to polymerise. The scaffold matrix may be allowed to polymerise before culture medium is added to the dish or well. This process results in the formation of domes of scaffold matrix in which the organoids grow. A typical scaffold matrix used for this purpose is BME, typically in a concentration of 80-100% (v/v) (not including the concentrated cell or organoid solution with which the matrix is mixed and which represents a negligible fraction of such concentrated scaffold matrix solution). The same solution can be used to coat the surface of the dishes or wells prior to deposition of the droplets of concentrated BME solution comprising the organoids or organoid precursor cells. Without wishing to be bound by theory, it is believed that such domes cannot be obtained using compositions as described herein, which contain much lower amounts of scaffold matrix. Indeed, droplets of the compositions described herein (or a solution comprising said composition and organoid progenitor cells or organoids) would not polymerise into a dome of scaffold matrix as the concentration of the scaffold forming polymers in the composition is too low to form a continuous scaffold. For short term culture such as e.g. drug screening, it is possible to deposit organoids onto a previously applied and polymerised layer of scaffold matrix. In such protocols, the organoids embed themselves in and/or sit on the matrix. Such protocols are not well suited for long term/large scale expansion of organoids because they either require very large amounts of scaffold matrix (in order to provide a polymerised layer of scaffold matrix over a surface area that is sufficient to support large cultures) and/or a very high number of single well plates each well comprising a polymerised layer of scaffold matrix. This is logistically and economically prohibitive.

By contrast, the present inventors have found that it is possible to culture organoids in suspension in a solution comprising much lower amounts of scaffold matrix, and in particular amounts that are not sufficient to form a continuous scaffold fixed to a surface of the container. Such solutions form small particles of scaffold matrix onto which the organoids or organoid progenitor cells can attach themselves. However, these small clusters are not fixed to a surface. While they can sediment under the action of gravity, especially as the organoids grow, they do not form a continuous structure affixed to the cell culture container and can be re-suspended mechanically, such as e.g. by agitation. The present inventors have surprisingly found that in this system, the organoids remain three-dimensional (i.e. they preserve the 3D organisation seen in scaffold culture and absent from adherent cultures without a scaffold), and can be grown and expanded for long periods of time. The inventors confirmed that the models have not been affected genomically through these alternate culturing conditions. In particular, the cultured models in the commonly used (dome-based) and new (suspension) conditions in parallel for up to 6 months and undertook both DNA and RNA sequencing at multiple timepoints. This confirmed that there was no significant genomic divergence. The inventors further proved that this system is suitable to conduct medium-high throughput perturbation screens that would be ergonomically and financially prohibitive in standard conditions. The inventors additionally proved that the organoids cultured in the new conditions were comparable to those culture in the standard organoid conditions (dome-based) in at least two different types of functional screens (CRISPR-Cas9 and drug perturbations).

Prior to the present invention, it was generally held that culture of organoids in domes of scaffold matrix, especially BME, was optimal, or even necessary at least for organoids derived from primary tissue. Indeed, organoids from primary (i.e. patient-derived) tissue typically thrive when the cells are maintained in close proximity. This is easily achieved using high concentrations or e.g. BME or Matrigel and small volume 3D cultures (as provided by the domes). Further, there was a widely held belief that complete encapsulation/embedding of the cells in an appropriate scaffold matrix was a requirement for organoid growth. The present inventors have surprisingly discovered that this is not the case, and that organoids can be grown attached to small particles of scaffold matrix in a culture medium.

This new approach has many benefits. Indeed, the use of scaffold matrix domes, while suitable for small or medium scale expansions, becomes practically unfeasible with large numbers of organoids. Indeed, this approach would require a high number of domes to be individually plated out, in either large plates or very high number of plates, the latter increasing the work intensive nature of the process and the former making the process highly susceptible to infection and/or contamination. The new approach therefore is advantageous from an ergonomics point of view since there is no need to individually deposit droplets—a task that is very delicate and typically done by manual pipetting. Such a task is therefore prone to generating problematic repetitive stress injuries. Further, the plated dome format of the conventional organoid culture system is particularly vulnerable to infections. By contrast, the culture of organoids in suspension in e.g. flasks carries a much lower risk of infection. Further, the new approach requires less than half of the amount of scaffold matrix that would be necessary to generate enough organoids for a screen such as a genome-wide CRISPR-Cas9 screen, using the conventional (dome-based) method. Scaffold matrices that efficiently support the growth or organoids are typically either biological materials extracted from cell cultures, or highly specialised synthetic materials. These materials are therefore costly to manufacture. Finally, the new approach also represents a significant time saving. Indeed, organoid passaging is quicker in new conditions compared to standard conditions as there is less scaffold matrix to digest prior to or during dissociation of the organoid structures, and no incubation time required for the scaffold matrix domes to polymerise before media can be added. While media change in the dome setting is arguably quicker as medium can be pipetted out and replaced without disrupting the domes, this is largely offset by the time gained in passaging, which is a more time consuming process in both approaches (such that time saving in this process has a disproportionate impact on the overall time saved). For example, the inventors estimated that using the new approach, organoid passaging time was reduced by approximately 20 to 30 minutes—primarily due to the reduced time required to digest old matrix and re-polymerise new matrix. By contrast, changing the medium is only a few minutes (approximately 5 minutes) longer with the new approach compared to the standard (dome) approach. As the skilled person understands, the exact amount of overall time saved may depend on various parameters such as e.g. the exact protocol used for medium change and passaging, and the frequency of passaging and medium change. For example, different organoid lines may require different frequency of passaging. In general, the more frequent the organoids have to be passaged, the more significant the time saving associated with the present approach will be.

In embodiments, the scaffold matrix is used at a concentration such that it forms particles in suspension in the composition. This is in contrast with the conventional dome-based approach in which the scaffold matrix is used in a concentration such that a continuous matrix structure is used (e.g. a dome of polymerised matrix), which is supported on a surface. Any of the concentrations described herein, such as e.g. a concentration that is equivalent to a concentration of between 2% (v/v) and 18% (v/v) of a complex protein hydrogel having a protein concentration between 12 and 18 mg/ml, may be such that the scaffold matrix forms particles in suspension in the composition. In particular, concentrations that are equivalent to a concentration of between 3% (v/v) and 15% (v/v) of a complex protein hydrogel having a protein concentration between 12 and 18 mg/ml may be particularly useful in compositions where the scaffold matrix forms particles in suspension in the composition. Therefore, also described herein is a composition comprising culture medium and particles of scaffold matrix in suspension in the culture medium. Aldo described herein are methods of culturing, screening or passaging organoids, in which a composition comprising culture medium and particles of scaffold matrix in suspension in the culture medium is used and optionally also prepared. In embodiments, the particles of scaffold matrix have a diameter of between a few hundred μm (such as e.g. 200, 300, 400, 500 μm) and approximately 3 mm. For example, the particles of scaffold matrix have a diameter of at least 200 μm, at least 300 μm, at least 400 μm or at least 500 μm, and at most 3 mm, at most 2.5 mm, or at most 2 mm. The diameter of a particle of scaffold matrix refers to the diameter of the smallest sphere that includes the particle. As the skilled person understand, particles may in practice show a distribution of sizes. As such, references to the sizes of the particles may refer to the median or average particle size, or to the range of size that includes at least a predetermined proportion (e.g. at least 50%, at least 60%, at least 70% or at least 80%) of the particles. For example, the particles of scaffold matrix have an average or median diameter between a few hundred μm (such as e.g. 200, 300, 400, 500 μm) and approximately 3 mm. As another example, at least 50%, at least 60%, at least 70% or at least 80% of the particles may have a size between a few hundred μm (such as e.g. 200, 300, 400, 500 μm) and approximately 3 mm. Further, the size of the particles and/or the distribution of sizes of particles may vary during a culture, for example as particles break up during manipulation. Without wishing to be bound by theory, the inventors believe that the exact size of the particles does not impact the success of the culture. Thus, the invention is not limited to any particle size that is compatible with the concentration ranges above where the cells do not grow attached to a continuous scaffold supported on the surface of a container.

Also described are an organoid culture comprising a population of organoids and a composition as described above, and a population of organoids that has been obtained using the methods for providing an expanded population of organoids described herein.

The organoid population may display long term stability. For example, the organoids may be maintained in culture for at least 6 months without significant genomic or phenotypic abnormalities. The organoids may be maintained in culture and expanded to a population comprising at least 10\hat{ }4, at least 10\hat{ }5, at least 10\hat{ }6 or at least 10\hat{ }7 individual organoids. Genomic abnormalities may be assessed at the genome level (e.g. by investigating mutations and/or copy number variations in the genomes of the organoids, for example using whole genome sequencing or whole exome sequencing), the transcriptome level (e.g. by investigating gene expression at the transcript level, for example using RNA sequencing, qRT-PCR, microarrays, etc.) or the proteome level (e.g. by investigating gene expression at the protein level, for example using mass spectrometry, fluorescence activated cell sorting, protein microarrays, dual modality sequencing such as e.g. CITE-seq, etc.). Phenotypic abnormalities may be assessed using functional screens, such as e.g. drug response screens, RNA interference screens, gene editing screens, etc.

The organoid population may display a long term stability that is similar to that of matched organoids (i.e. organoids obtained from the same population or progenitor cells and maintained in culture for a similar amount of time) cultured in standard (dome-based) organoid culture conditions. For example, the organoids may show a pattern of gene expression and/or an amount of genomic alteration accumulated over time in culture that is comparable to those of matched organoids cultured in standard (dome-based) organoid culture conditions. The organoids may show a functional long term stability that is similar to that of matched organoids cultured in standard (dome-based) organoid culture conditions. For example, the organoids may show a similar response in functional screens compared to that of matched organoids cultured in standard (dome-based) organoid culture conditions.

Using the approaches described herein, the organoid culture can be maintained for at least two passages, at least 5 passages, at least passages. Using the approaches described herein, the organoid culture can be maintained for more than two, three, four, five, six, seven, eight, nine, ten weeks, 20 weeks from seeding.

Using the approaches described herein, organoid organisation may be preserved after two, three, four, five, six, seven, eight, nine, ten weeks, 20 weeks from seeding.

As used herein, “seeding” refers to the act of preparing a culture of individual cells or substantially individual cells from which organoids can be derived (i.e. organoid progenitor cells), for culturing in conditions supporting organoid growth.

As used herein, “passage” or “passaging” is the act of transferring some or all cells from a culture to a fresh culture medium, in order to reduce the concentration of cells in the culture. Passaging may also be referred to as subculturing. Passaging is an important part of an expansion process as it enables the culture to grow in higher numbers. By contrast, in a culture medium change, the concentration of the cells or organoids in the culture is typically not altered in a significant manner.

Also described is a method of passaging or changing the medium in an organoid culture, comprising:
(i) providing a cell culture as described herein comprising a population of organoids, a culture medium and a scaffold matrix;
(ii) centrifuging the cell culture to obtain a pellet comprising the organoid population and a supernatant, for example at 400 g, 500 g, 600 g, 700 g, 800 g or 900 g (preferably 800 g) for about 60 seconds, 90 seconds, 120 seconds, 150 seconds or 180 seconds (preferably 120 second/2 min);
(iii) optionally disrupting the organoids, preferably
enzymatically and/or mechanically, such as e.g. by exposing the organoids to a trypsin or TryplE™ solution (for example between and 10 minutes) then pipetting the solution at least once to mechanically separate the cells;
(iv) mixing the (optionally disrupted) organoids with a composition as described herein comprising a culture medium and a scaffold matrix, thereby producing a passaged or medium changed organoid culture.

Disrupting the organoids may be performed in order to produce a population of isolated organoid progenitor cells. Organoids may be disrupted mechanically, enzymatically and/or chemically. For example, organoid progenitor cells may be obtained by digesting the scaffold matrix (either added in culture or present in a primary tissue sample), harvesting the cells or organoids by centrifugation, and disrupting the pellet thus obtained into individual cells, for example mechanically. The individual progenitor cells thus obtained may be re-suspended and cultured as described above in the composition comprising a culture medium and scaffold matrix, where they (re)form into organoids. When the scaffold matrix is a complex protein hydrogel, a proteolytic enzyme or enzyme mixture may advantageously be used to digest the scaffold matrix. For example, trypsin or TryplE™ may be used.

Mixing the pellet with a composition as described herein may comprise mixing the pellet with a culture medium and adding the scaffold matrix to the composition comprising the organoids and the culture medium.

Also described are methods of screening an organoid or a population of organoids comprising: contacting an organoid or population of organoids with a test compound; and determining the effect of the test compound on the organoids or population of organoids, wherein the organoids or population of organoids were obtained using the methods described herein, and/or wherein the contacting is performed while the organoids are in suspension in a composition as described herein.

The contacting may be performed while the organoids are in suspension in a composition comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml. Alternatively, the contacting may be performed while the organoids are in suspension in a composition comprising a culture medium and a scaffold matrix, wherein the scaffold matrix is present in the composition at a concentration that is at least half of the above-described concentration. For example, such a composition may be obtained by culturing the population in a composition comprising a culture medium and a scaffold matrix as described herein for a first period of time, and culturing the population in a composition comprising a culture medium and a scaffold matrix in a concentration that is at least half of that of the concentrations described herein for a further period of time. In such cases the contacting may be performed during the first or further period of time.

Screening may refer to drug screens, gene editing (e.g. CRISPR-Cas9) screens, or RNA interference screens (e.g. shRNA screens). As such, the test compound may for example be a drug, a CRISPR-Cas9 guide RNA, or an interfering RNA.

The proliferation, growth, apoptosis or viability of the organoids, protein production, metabolic activity of key enzymes, expression of one or more genes (such as e.g. stress response genes), or the ability of the organoids to perform one or more cell or organoid functions may be determined in the presence relative to the absence of the test compound. For example, a decrease in proliferation, growth, viability or ability to perform one or more cell or organoid functions may be indicative that the compound has a toxic effect. Conversely, an increase in growth, viability or ability to perform one or more cell or organoid functions may be indicative that the compound has a beneficial effect on the organoids.

Also described herein is a kit for the production of expanded populations of organoids comprising a composition as described herein or a culture medium (or equivalent amount of concentrated medium) and a scaffold matrix in the relative amounts described herein. Where equivalent amounts of a concentrated medium are provided, the kit may further comprise instructions providing the amount of liquid (e.g. water, distilled water, sterilised water, buffer) to be added to the concentrated medium to obtain the appropriate amount of culture medium. The kit may further comprise one or more cell culture containers, such as plates or flasks. Preferably, the cell culture containers are low adherence cell culture containers.

Other aspects and embodiments of the invention provide the aspects and embodiments described above with the term “comprising” replaced by the term “consisting of” and the aspects and embodiments described above with the term “comprising” replaced by the term “consisting essentially of”. Modifications of the above embodiments, further embodiments and modifications thereof will be apparent to the skilled person on reading this disclosure, and as such, these are within the scope of the present invention. Any sub-titles herein are included for convenience only, and are not to be construed as limiting the disclosure in any way. The following is presented by way of example and is not to be construed as a limitation to the scope of the claims.

### EXAMPLES

### Materials and Methods

### 1. Organoid Culture

For standard droplet culture organoids are suspended in 20μl 80:20 ECM:Media droplets with a protein concentration of between 6.4-9.6 mg/ml (BME, Amsbio 3533-005-02), on dried pre-warmed 6 well plates with previously published media recipes (8, 15). The droplets are incubated at 37° C. for 20 minutes, when 2 ml media is added to each well. For the 5% ECM technique, organoids are mixed with the same volume of media as would have been used in the droplet condition, e.g. for 1 well of a 6 well plate take 2 ml media, add 5% volume ECM (100 ul BME-2) and immediately transfer to an ultra-low adherent plate or flask and place in an incubator.

Ultra-low adherent plates were obtained from Corning™ (Costar™ 6-well clear flat bottom ultra-low attachment plates, product number 3471—these plates include a covalently bound hydrogel layer that inhibits cellular attachment). Low attachment T75 culture flasks from Corning™ (product number 3814—these flasks include a covalently bound hydrogel layer that inhibits cellular attachment) were used as ultra-low adherent flasks.

Cell-repellent plates were obtained from Greiner™ (Cellstar™ 6-well clear plates with cell repellent surface, product number 657970—these plates have a chemically modified polymeric surface that prevents cell adhesion).

“Conventional” plates were obtained from Corning™ (Costar™ 6-well clear TC-treated flat bottom plates, product number 3516—these plates have been “tissue culture-treated” to improve cellular attachment, by exposing the surface to a plasma gas to make the polymer more hydrophilic).

### 2. Organoid Media

The compositions used are described in Tables 1 and 2.

TABLE 1
Oesophageal media components

| Reagent | Volume | |
|---|---|---|
| Advanced DMEM F12++ supp | 107 | ml |
| WNT3A CM (50%) | 200 | ml |
| R-Spondin-1 CM (20%) | 80 | ml |
| B27-supplement | 8 | ml |
| Nicotinamide | 4 | ml |
| N-acetyl cysteine (mix if ppts) | 1 | ml |
| Recombinant Noggin | 400 | μl |
| Recombinant Human EGF | 40 | μl |
| A83-01 | 40 | μl |
| SB202190 | 40 | μl |
| FGF-10 | 80 | μl |

TABLE 2
Colon media components.

| Reagent | Volume | |
|---|---|---|
| Advanced DMEM F12++ supp | 307 | ml |
| R-Spondin-1 CM (20%) | 80 | ml |
| B27-supplement | 8 | ml |
| Nicotinamide | 4 | ml |
| N-acetyl cysteine (mix if ppts) | 1 | ml |
| Recombinant Noggin | 400 | μl |
| Recombinant Human EGF | 40 | μl |
| A83-01 | 40 | μl |
| SB202190 | 40 | μl |
| PGE2 | 40 | μl |
| Gastrin | 40 | μl |

### 3. Whole Genome Sequencing & Analysis

Whole genome 150 base paired-end sequencing reads were generated using Illumina HiSeq X Ten platform. Reads were aligned using Burrows-Wheeler Alignment (BWA-MEM) tool (10). PCR duplicates, unmapped and non-uniquely mapped reads were filtered out before downstream analysis. Single base substitutions and indels were identified using CaVEMan (11) and cgpPindel (12) respectively. Germline variants and technology-specific artefacts were removed by filtering against a matched normal blood sample and the panel of 100 unrelated normal samples (ftp://ftp.sanger.ac.uk/pub/cancer/dockstore/human/SNV_INDEL_ref_GRC h37d5.tar.gz). Additional post-processing filters were applied using in house post-processing tool cgpCaVEManPostProcessing (https://github.com/cancerit), variants sites that were flagged as ‘PASS’ were considered for further downstream analysis. Unbiased analysis of mutant and wild-type reads found at the loci of the base substitutions and indels were assessed across the related samples using vafCorrect (13). For the copy number variation (CNV) analysis, genome wide segmented copy number log R data was derived using ascatNgs (Raine et al., 2016) which uses the Allele Specific Copy Number Analysis of Tumors (ASCAT) algorithm (Van Loo et al., 2010). ASCAT segments overlapping with summary intervals were merged and mean log R of merged segments was assigned to a given summary interval and to the underlying genes in that interval. These intervals were used for further downstream analysis. Copy number states of these genes were used for downstream analysis. Driver genes with diploid status (log R=0) were removed from the copy number tile plot.

### 4. RNAseq & Analysis

Paired-end transcriptome reads were quality filtered and mapped to GRCh37 (ensemble build 75) using STAR-v2.5.0c (14) with a standard set of parameters (https://github.com/cancerit/cgpRna). Resulting bam files were processed to get per gene read count data using HTSeq 0.7.2. We calculated TPM (Transcripts Per Million) values using the count and transcript length data for further downstream analysis. Only ‘protein coding’ genes (22810) were considered for downstream QC and filtering steps. We used ‘filterByExpr’ (EgdeR)(28) function with cutoff of 10 and 100 for min.count and min.total.count respectively with min.prop cutoff of 90%, resulting filtered genes (15793) were used for sample level correlation analysis. Gene level clustering was performed on top 8500 variance ranked genes.

### 5. Drug Screening

Four of the lines from the longitudinal study were assayed in technical triplicate and biological duplicate for drug response in a 3 day viability assay with 72 individual drugs at T2. Formed organoids are seeded into 384 well plates onto a layer of Basement Membrane Extract using a XRD-384 (Fluid-X) reagent dispenser. Compounds are screened using a 7-pt dose response curve with a half-log dilution series covering a 1000 fold range. The dosing of the compounds is carried out using an Echo 555 (Labcyte) acoustic dispenser and the duration of drug treatment is 72 hours (3 days). Cell viability is measured using CellTitre-Glo 2.0 (Promega) reagent. Dose response curves were fitted to experimental data using a non-linear mixed effects model (29). The organoid model, the % BME media condition, and the drug treatment were included as random effects. Fitted curves with an RMSE of greater than 0.3 were removed from the data. Compound activity was calculated as 1−AUC, where the AUC is the area under the curve within the screened dose range, thus the activity range is measured from 0 (no activity) to a maximum of 1. Analysis of variance was used to assess the effect of the organoid model and ECM condition on the activity of each compound, i.e. a linear model with the form: compound˜% ECM+organoid model.

### 6. Whole-Genome CRISPR Screening

Stable Cas9 expressing lines were generated using lentiviral transduction and antibiotic selection at day 6. gRNA library transduction was performed at 100× coverage of the Human CRISPR Library v.1.1 with an MOI of 0.3, and following library transduction lines were cultured for 3 weeks. All plasmids and sample processing post library harvest were as per Behan et al, 2019 (9). Blasticidin was used for selection of Cas9 positive cells. The optimum concentration of blasticidin (minimum concentration required to kill wild type cells) for each organoid line was determined by culturing cells suspended in media+5% BME+various blasticidin concentrations (0-75 μg/ml final concentration) in 96 well plates for 72 h and using the CellTitre-Glo 2.0 (Promega) reagent to assess viability. CRISPR-Cas9 screens analysis was performed similarly to Goncalves et al, 2020 (25). Briefly, these started from sgRNA read count matrices. For each sample the number of sgRNAs with at least 10 counts was calculated to evaluate the library representation. Read counts were normalised to reads per million within each sample. Log 2 fold-changes were calculated compared to the plasmid DNA (pDNA). Lastly, gene-level fold-changes are calculated by taking the mean fold-change of all targeting sgRNAs. Replicates were merged by averaging the gene-level fold-changes. Recall curves of essential and non-essential genes (26, 27) are estimated by ranking all the genes ascendingly according to their gene-level fold-change and the cumulative distribution is calculated. This is then summarized by estimating the area under the recall curve, where areas over 0.5 (random expectation) represent enrichments towards negative fold-changes, and areas lower than 0.5 represent enrichment towards positive fold-changes.

### 7. Organoid Passage and Media Change in 5% BME in Flasks

The following materials may be used in the protocols below: Phosphate buffered saline (DPBS) (e.g. Life Technologies #14190-094); Advanced DMEM/F-12-500 mL (e.g. Life Technologies #12634010) TrypLE Express (e.g. Life Technologies #12604021); Complete culture media (see above); Via 1 cassettes (e.g. 941-0012—Chemotec); RGF BME, type 2 (e.g. Cultrex 3533-005-02); Low attachment T75 culture flask (e.g. Corning 3814); 10 μg/ml Blasticidin (optional) (e.g. Invivogen ant-bl-1); 1 mg/ml puromycin (optional) (e.g. Invivogen ant-pr-1 diluted 1 in 10).

Organoid lines are preferably only manipulated once every week including media changes.

To allow organoids to fully recover from being broken down, it is preferable to passage organoids just once every 2 weeks, although in some cases lines may be passaged weekly. If passaged within the week, the line may not require a media change. If media is spent quickly following a media change/passage, either re-suspend the line in additional media, or expand lines into extra labware on subsequent passages. If lines have already been manipulated within the week, or are due to be passaged/broken down later in the week, a top up media change may be preferred. Flasks can be topped up with a relevant amount of complete media (including antibiotic), without the addition of BME2, if required.

### A. Transfer to Flask

i. Harvest whole organoids from entire plate, incubating in TryplE for a maximum of 10 minutes to remove any BME2 from the culture.
ii. Centrifuge at 800 g for 2 min and aspirate supernatant.
iii. Suspend the pellet in at least 1 ml of complete media, and mix well by pipetting with a P1000 to ensure any aggregates and clumps are completely broken down.
iv. Using a 5 ml stripette, seed cell suspension into ultra-low adhesion (ULA) flask with an appropriate amount of media (and optionally antibiotic) depending on split ratio chosen and the volume used to re-suspend the pellet (see Table 3 below). When transferring to flasks from x1 6 well plate, do not seed into a total volume higher than 20 mls (roughly a 1:2 split ratio) as lines may take time to adapt to the new culture conditions.

TABLE 3
Amounts of medium and BME to be used when
transferring organoids to 5% BME flasks

| | Total | | | | Volume of | Volume of |
|---|---|---|---|---|---|---|
| Split ratio | volume | | Volume of | | Blasticidin | Puromycin |
| (approximate) | of media | | BME2 | | (ul) | (ul) |
| 1:1 | 11.4 | ml | 0.6 | ml | Concentration | Concentration |
| 1:2 | 19 | ml | 1 | ml | (ug/ml) × Total | (ug/ml) × Total |
| | | | | | volume × 0.1 | volume |

For antibiotic volumes using Table 3, see the following examples: Blasticidin concentration of 25 ug/ml in a total volume of 12 mls (25×12×0.1=30 ul); Puromycin concentration of 3 ug/ml in a total volume of 20 mls (3×20=60 ul).
v. Add appropriate amount of BME2 (see Table 3) to the flask suspension using a stripette and mix very well by pipetting and incubate at 37° C., 5% CO₂.
vi. Following transfer, the cell line can be left for up to 1 week without manipulation. Inspect media colour over the days following flask transfer, if line appears to be growing quickly and the media is spent; either a complete media change/expansion passage may be required within the first week (see procedure below).

### B. Flask Passage

i. Remove flask from incubator and check for organoid density/size as well as media colour (is media spent?). Do not allow organoids to get too dense, crowded etc.
ii. Collect suspension culture in either 15 ml or 50 ml falcon tubes using a stripette or by pouring suspension.
iii. Wash the flask/s with 5-10 ml of Advanced DMEM using a 10 ml stripette and add to the already collected suspension. Mix the suspension well by pipetting to break down any larger clumps of BME2/aggregates before centrifugation.
iv. Centrifuge at 800 g for 2 min. Aspirate the supernatant.
v. Once aspirated, suspend the pellet in an appropriate amount of TryplE depending on the size of the pellet. Small pellets can be suspended in up to 10 ml, whilst larger pellets may need to be suspended in up to 40 ml per falcon tube.
vi. Mix well by pipetting and place in a 37° C. water bath.
vii. Check organoid suspension under the microscope after 5 minutes and then as required to assess and monitor the dissociation of the organoids. Use a P1000 to pipette the cell suspension up and down to help dissociate the organoids.
viii. Centrifuge at 800 g for 2 min. Aspirate off supernatant to leave organoid cell pellet.
ix. Depending on the pellet size; suspend in a minimum of 1 ml complete media and mix well by pipetting with a P1000 to ensure any aggregates and clumps are completely broken down.
x. Using a 5 ml stripette, seed cell suspension into ULA flask with an appropriate amount of media and antibiotic (if used) in the flask already depending on the split ratio chosen (see Table 4 below for examples of flask volumes).
xi. Add appropriate amount of BME2 (see Table 4) to the flask suspension using a stripette and mix very well by pipetting and incubate at 37° C., 5% CO₂.

TABLE 4
Amounts of medium and BME to be used when
passaging organoids between 5% BME flasks

| | Total volume | | | | | |
|---|---|---|---|---|---|---|
| Total | of media | | | | Volume of | Volume of |
| volume | (including cell | | Volume of | | Blasticidin | Puromycin |
| per flask | suspension) | | BME2 | | (ul) | (ul) |
| 12 ml | 11.4 | ml | 0.6 | ml | Antibiotic | Antibiotic |
| 20 ml | 19 | ml | 1 | ml | concentration | concentration |
| 30 ml | 28.5 | ml | 1.5 | ml | (ug/ml) × Total | (ug/ml) × Total |
| 40 ml | 38 | ml | 2 | ml | volume × 0.1 | volume |
| 50 ml | 47.5 | ml | 2.5 | ml | | |
| 60 ml | 57 | ml | 3 | ml | | |

For antibiotic volumes using this table, see the following examples: Blasticidin concentration of 25 ug/ml in a total volume of 12 mls (25×12×0.1=30 ul). Puromycin concentration of 3 ug/ml in a total volume of 20 mls (3×20=60 ul).

### C. Media Change

i. Collect suspension culture in either 15 ml or 50 ml falcon tubes using a stripette or by pouring suspension.
ii. Mix the suspension well by pipetting to break down any larger clumps of BME2/Aggregates before centrifugation.
Because suspension will be seeded back into original labware, the Advanced DMEM wash step is not necessary when collecting the culture.
iii. Centrifuge at 800 g for 2 min. Aspirate the supernatant.
iv. Depending on the pellet size; suspend in a minimum of 1 ml complete media and mix well by pipetting with a P1000 to ensure any aggregates and clumps are completely broken down.
v. Using a 5 ml stripette, seed cell suspension into ULA flask with an appropriate amount of media and antibiotic in the flask already depending on the split ratio chosen (see Table 4 above for examples of flask volumes).
vi. Add appropriate amount of BME2 (see Table 4) to the flask suspension using a stripette and mix very well by pipetting and incubate at 37° C., 5% CO₂.

### 8. Histology and IHC

Following BME-2 dissociation, organoids were gently pelleted and fixed in 10% neutral formalin before paraffin embedding and sectioning. Paraffin embedded sections of 3.5 μm were stained by a Bond Max autostainer according to the manufacturer's instruction (Leica Microsystems). Primary antibodies cytokeratin (AE1/AE3, 1:100, Dako), Vimentin (D21H3, 1:100, Cell Signaling Technology), and p53 (D07, 1:50, Leica) were applied with negative controls as previously described (8).

### Example 1: Identification of an Alternative Large-Scale Organoid Expansion Technique

In order to address the ergonomic and cost implications of culturing organoids at scale several alternative techniques were investigated. These included 100% ECM droplets suspended in media and cultured in spinner flasks (4), microcarrier beads in a 5% ECM solution and an ECM concentration gradient in ultra-low attachment plates. For all techniques tested, published organoid media recipes were used. Spinner flasks and microcarrier beads failed to support organoid growth and did not improve the ease of handling large cultures. It quickly became evident that lower percentages of ECM (reduced matrix conditions) appear to address the necessary requirements most effectively, as indicated in Table 5 below which shows the characteristics of each of the 3 alternative methodologies tested, compared to the standard organoid culturing technique.

TABLE 5
Characteristics of alternative organoid expansion techniques

| | Supports | | | |
|---|---|---|---|---|
| | organoid | | Time | Cost |
| Technique | growth | Scalability | (benefit) | (benefit) |
| 80% BME | Yes | ++ | + | + |
| droplets | | | | |
| Reduced | Yes | +++ | +++ | +++ |
| matrix | | | | |
| conditions | | | | |
| Microcarriers | Yes | + | + | + |
| Spinner | No | | | |
| flasks | | | | |

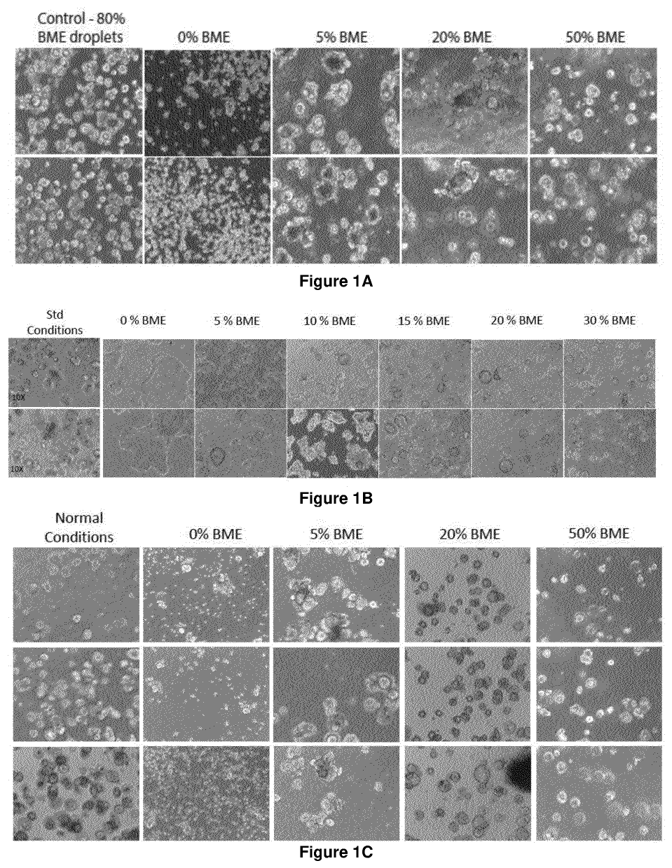

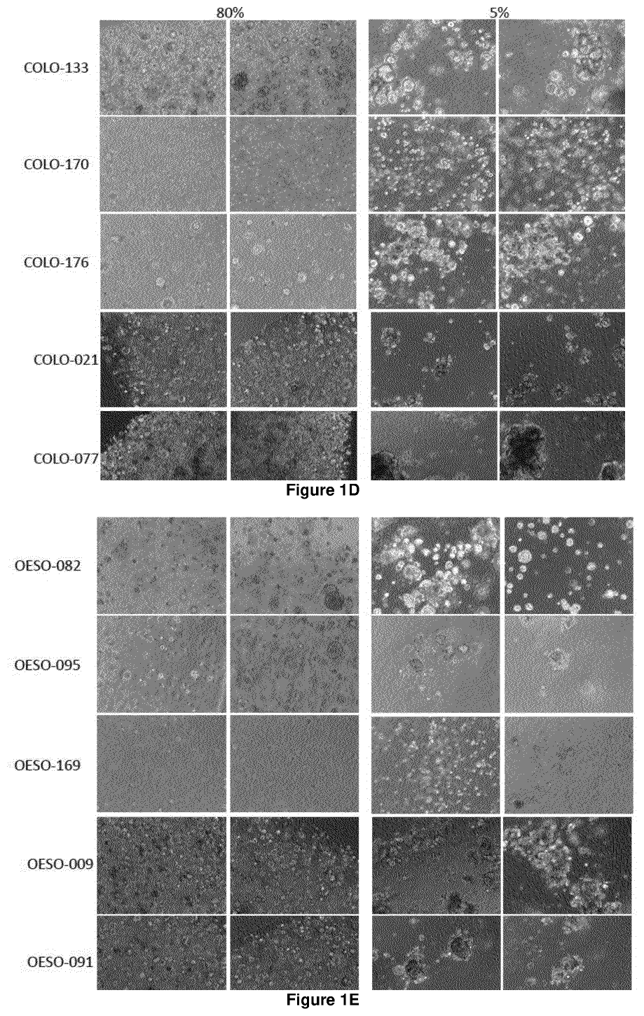

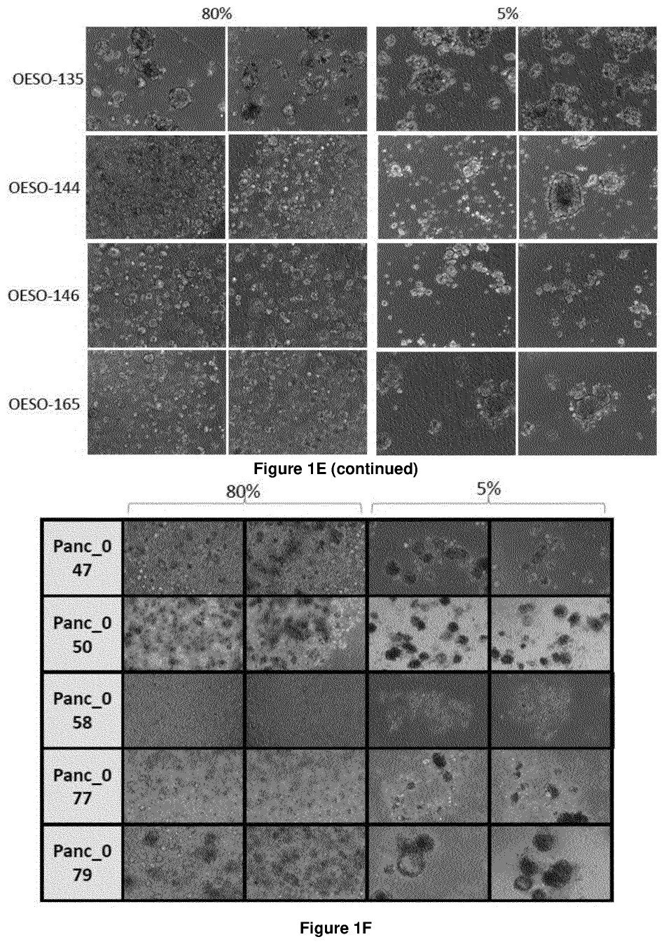

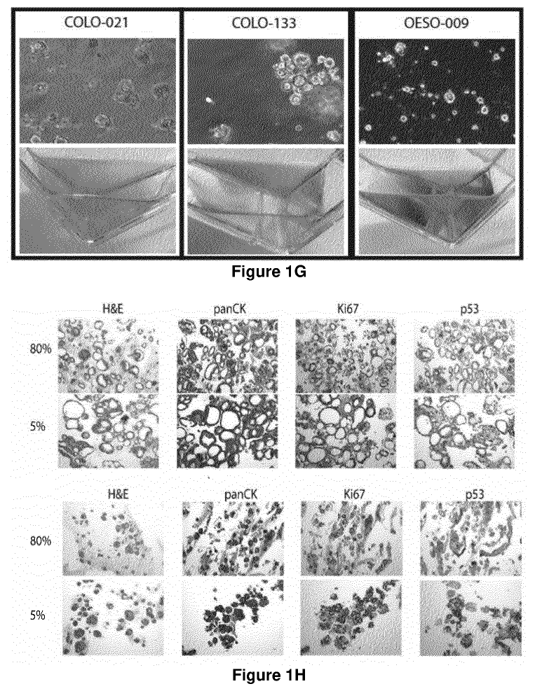

A matrix gradient ranging from 0-50% ECM was trialled in a colorectal organoid model (HCM-SANG-0266-C20, labelled as “COLO-005” below) both in standard and ultra-low attachment 6-well plates. In 0% ECM organoid formation was observed, but the culture was dominated by a loss of cell viability. Using 0-10% ECM (>0, <=10%, as evidenced by 5% and 10% ECM data points) resulted in the organoids growing as a suspension culture, attaching to pieces of ECM that had polymerised in the organoid media. Concentrations beyond 20% ECM led to complete polymerisation of the ECM and organoid media forming a solid ECM layer. Thus, 20-50% ECM resulted in organoids growing more akin to standard, fully-polymerised, solid culture conditions. The solid ECM layer formed using >20% ECM did not facilitate easier handling or significantly reduce the volume of ECM and associated cost compared to standard conditions. Short term organoid formation and growth was supported for a week in as little as 5% BME in ultra-low attachment (ULA) plates (see FIG. 1A which shows representative images of the organoids after 6 days in various BME conditions tested—using ULA plates, FIG. 1B which shows representative images of the organoids after 6 days in various BME conditions tested—using conventional plates, and FIG. 1C which shows representative images of the organoids after 6 days in various BME conditions tested—using plates with a cell repellent surface). Ultra-low attachment plates supported the formation of typical organoid structures. Conventional cell culture plates led to the organoids adhering to the bottom of the plate, which was considered suboptimal. All further optimisation experiments were conducted in ultra-low attachment plates and flasks. Cell repellent plates were found to be equally suitable. Based on these studies, 5% ECM was taken forward for further evaluation, although concentrations as low as 2.5% were also tested and found to be suitable (see Example 5).

To evaluate whether 5% ECM supported the formation and expansion of a range of organoid models a further 4 colorectal, 8 oesophageal and 5 pancreatic adenocarcinoma organoid models were successfully cultured for over one week (n=18 models), demonstrating the wider application of this approach in different tumour types. In total, short-term expansions in 5% ECM were shown to support organoid growth in 5 colorectal, 9 oesophageal and 5 pancreatic organoid models over one week (see FIGS. 1D-F). Culture was successful both in low adherence plates and flasks, and for all lines of organoids (see FIG. 1G which shows examples of cultures of colorectal and oesophageal organoids in plates and flasks), demonstrating the general applicability of the method. Organoids when grown in 5% ECM generally appeared to be larger in comparison to standard 80% organoid culturing techniques, also visible by H&E staining (see FIG. 1H), possibly due to not being confined to a 20 μl dome of ECM. The lack of confinement also led to an increased tendency for individual organoids to adhere together, again leading to the formation of large organoids. Initially, organoids appeared to have an increased tendency to adhere together. This reduced as experience with the technique increased, leading to better dispersion of the single cells within the solution and increased speed of completing the passaging task. Apart from being larger in size when grown in 5% ECM, no morphological differences were observed by H&E staining when compared to the counterpart models grown in standard conditions (FIG. 1H). Furthermore, Ki67 and p53 expression patterns were consistent in both culture conditions.

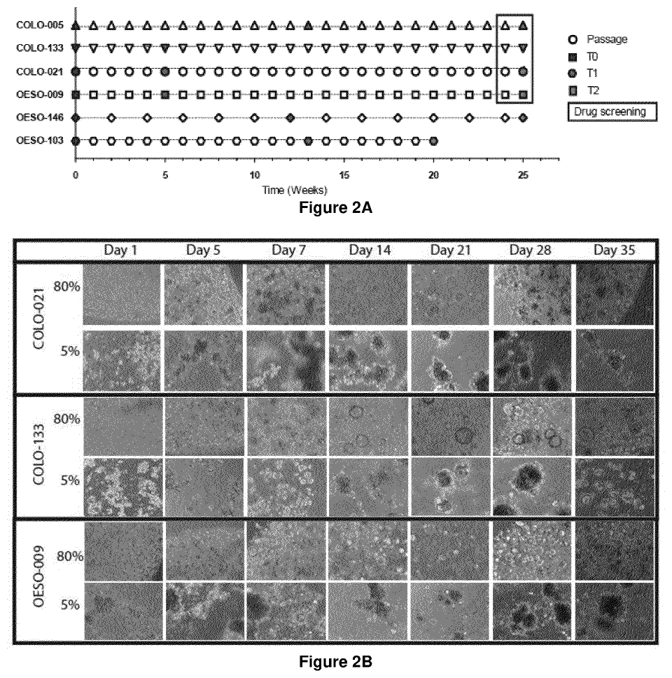

In order for the 5% ECM to be applicable for large scale expansion of hundreds of organoids, prolonged culture over multiple passages is required. To determine if the 5% ECM culture could support long-term organoid expansion, six models were cultured in parallel for up to 6 months in standard and 5% ECM conditions. This timeline was chosen to reflect the time that models may need to be in culture to perform high throughput screens. During this time they were subjected to genomic and phenotypic characterisation to ensure the culture conditions had little to no impact on genomic evolution in culture, as well as observed drug and gene dependencies. FIG. 2A details how these models were interrogated throughout this 6 month prolonged culture, at time point 0 (TO), time point 1 (T1, approximately 1-3 months) and time point 2 (T2, 6 months). FIG. 2B shows images of 3 organoid lines over the first 5 weeks of prolonged culture. The appearance of these 5% cultures over the first 7 weeks of the study are shown in FIG. 1c. We can clearly see that while the size of the organoids is consistently larger in the 5% ECM, the morphology of the organoids in each model remains stable over time in each culture condition. This remained stable for the duration of the entire longitudinal experiment. Also shown in FIG. 1G is the appearance of the 5% ECM culture when maintained in an ultra-low adherence flask rather than multi-well plates.

Together, this data demonstrates that the culture of organoids in suspension in medium comprising low percentages of BME is possible, even over long time scales.

The low (e.g. 5%) ECM suspension culture method provides multiple technical benefits over standard organoid culturing protocols, including a decreased time requirement per passage, less physical handling of ECM to reduce injury risk due to repetitive motions, and lower volumes of ECM equating to a significant reduction in cost. This technique can be scaled to use ultra-low adherent flasks, rather than multi-well plates, minimising incubator space requirements as well as reducing the risk of microbial and cross-model contamination.

### Example 2: Genomic Characterisation of Organoids in Suspension Culture

In vitro disease models are known to evolve over time in culture (5). Indeed, models acquire new mutations via intrinsic and extrinsic mechanisms (e.g. extrinsic selective pressures such as culture conditions). Within polyclonal organoid cultures it is possible that competition between different clones could contribute to this evolution. In characterising the 5% ECM culture method it was important to ensure that the culture conditions were not directly contributing to or adversely influencing how the models evolve while in culture. In order to assess this during the prolonged expansion (i.e. long-term culturing over a period of ˜6 months) six models were subjected to whole genome sequencing (WGS) and RNA sequencing (RNAseq) at TO, T1 and T2 following parallel culture in both 5% ECM and standard conditions (see FIG. 2A).

The inventors first took a global view of all variants identified in all samples, to assess whether there were any fundamental differences between the initial culture at TO and any time points.

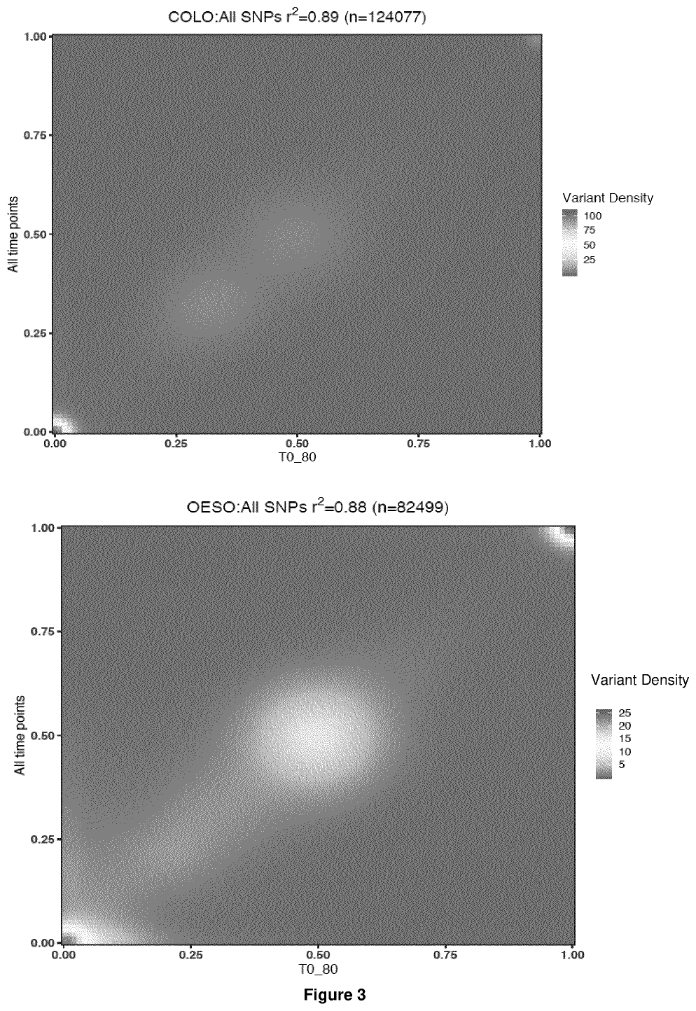

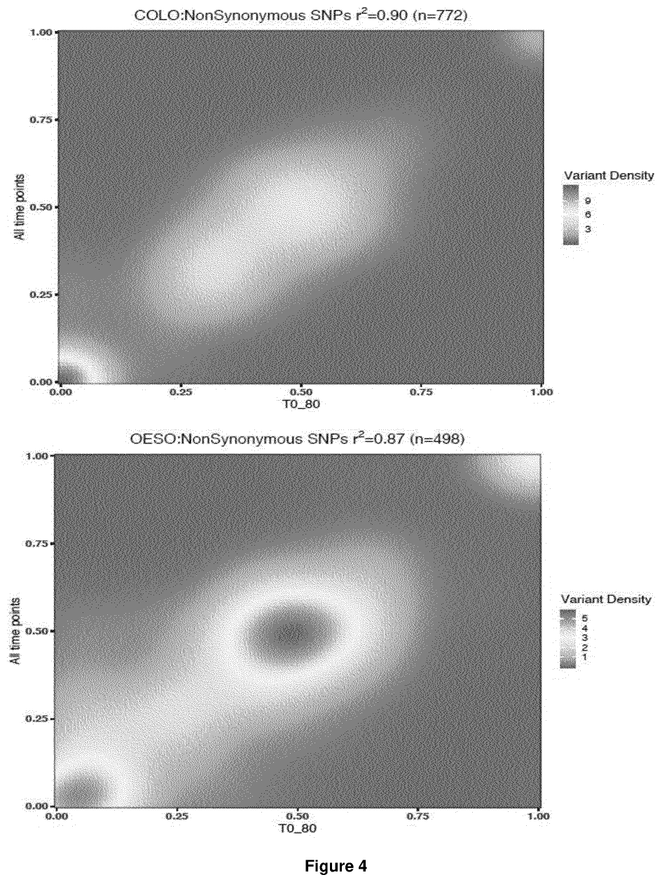

They began by comparing all single nucleotide polymorphisms (SNPs) identified in all samples. The results of this analysis are shown on FIGS. 3 and 4. These figures show correlation density plots of the VAF (variant allele frequency) for all variants (synonymous and non-synonymous SNPs—FIG. 3) and only the non-synonymous variants (FIG. 4), between TO and all other time points for 3 colon samples (FIGS. 3 and 4, left) and 3 oesophageal samples (FIGS. 3 and 4, right). The density plots displaying all SNPs (FIG. 3) and non-synonymous SNPs only (FIG. 4) are both highly correlated. In particular, the r² for the colon samples across all variants (n=124,077) was 0.89 (FIG. 2. left); the r² for the oesophagal samples across all variants (n=82,499) was 0.88 (FIG. 2, right); the r² for the colon samples across non-synonymous variants (n=772) was 0.90 (FIG. 3, left); and the r² for the oesophagal samples across non-synonymous variants (n=498) was 0.87 (FIG. 3, right).

Thus, the variant allele fraction (VAF) of SNPs, irrespective of time point or culture condition, were highly correlated with TO in standard conditions when considering all SNPs (FIG. 3) or non-synonymous SNPs in colon or oesophageal models (FIG. 4). This data indicates that the variant allele fraction (VAF) of the vast majority of variants identified across the samples remains consistent.

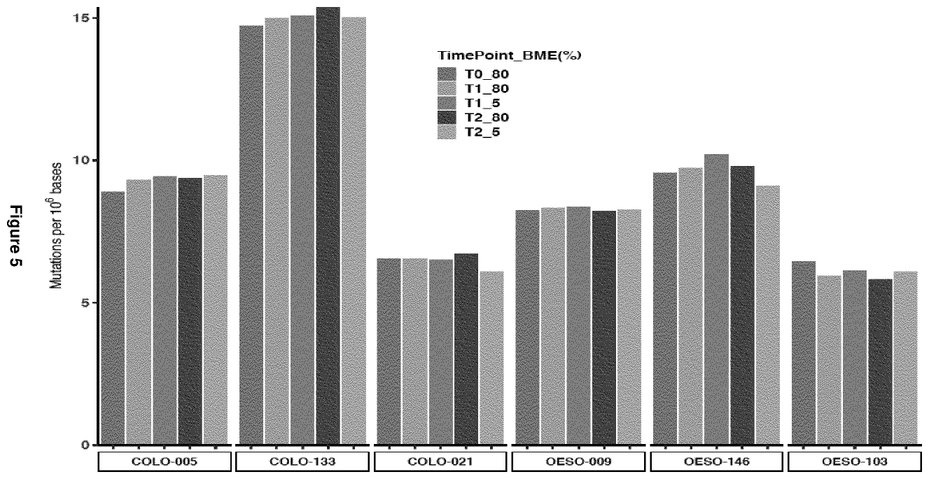

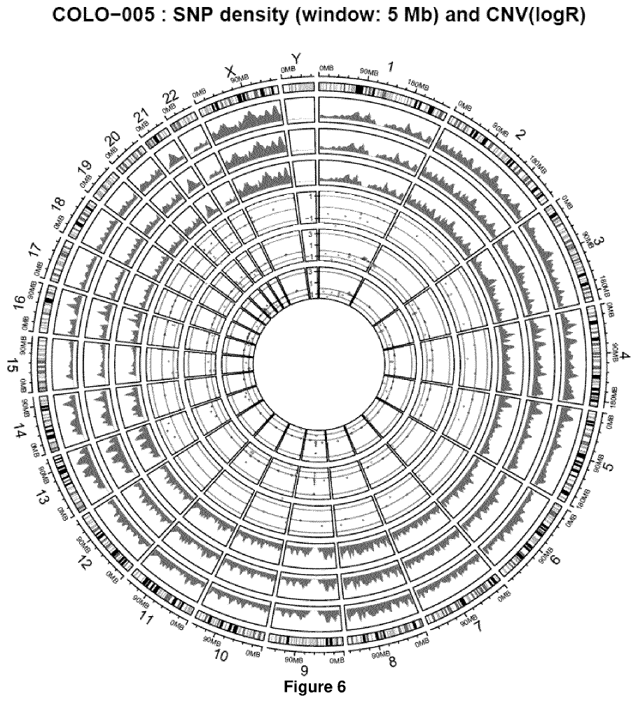

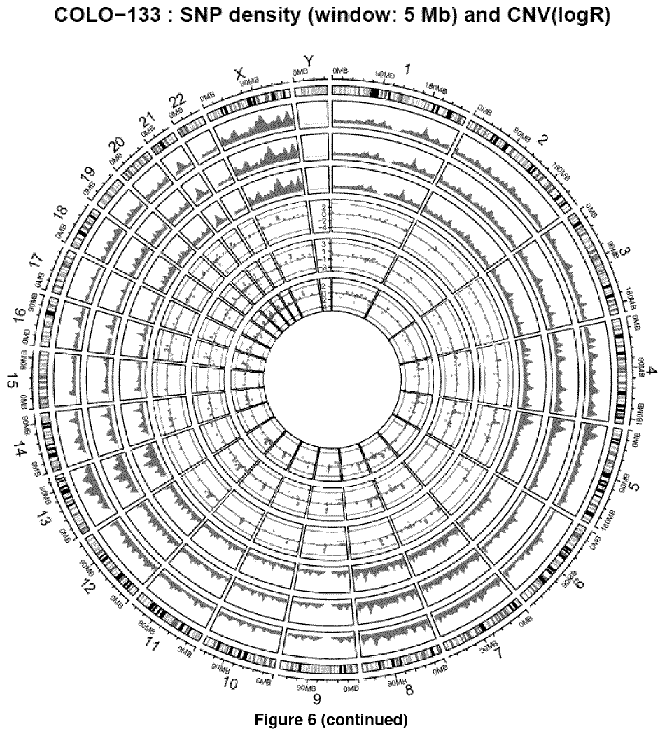

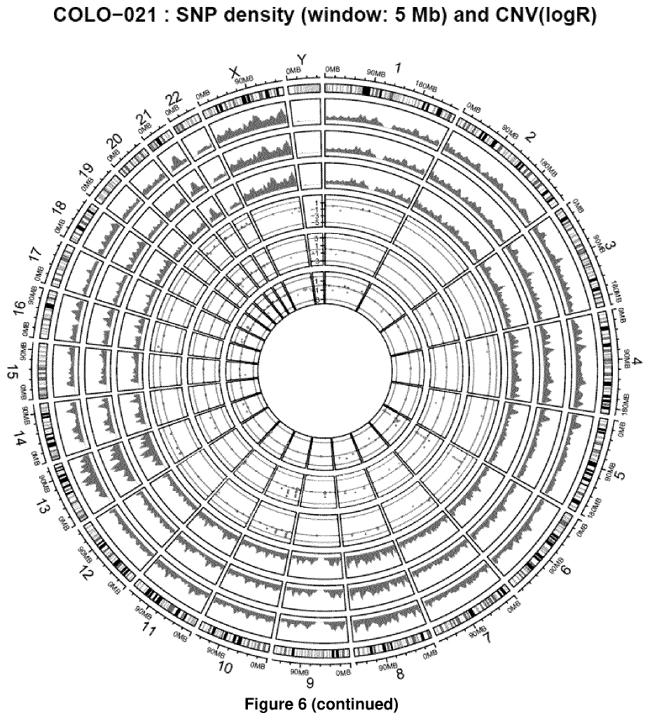

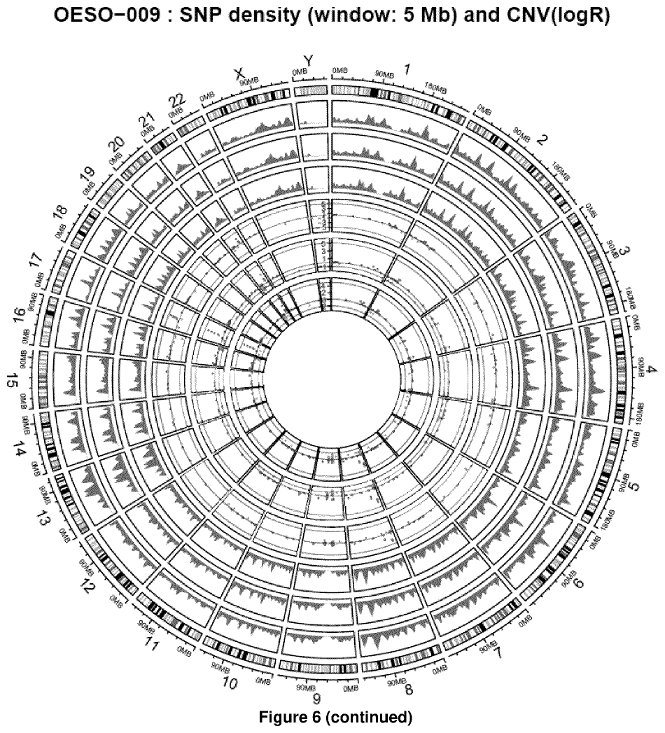

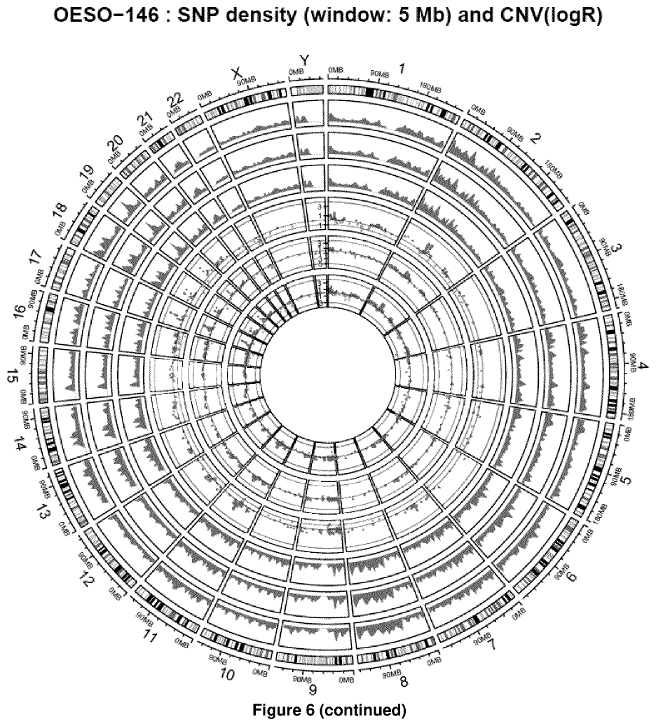

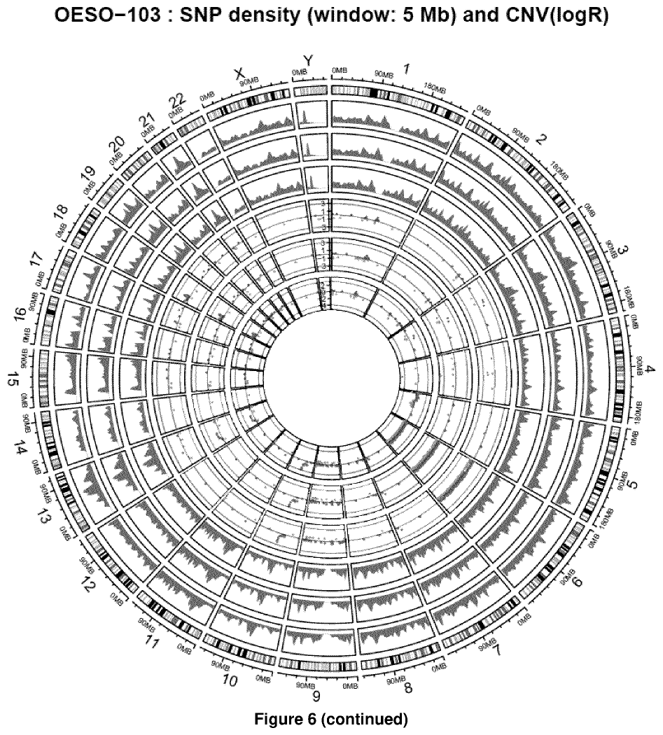

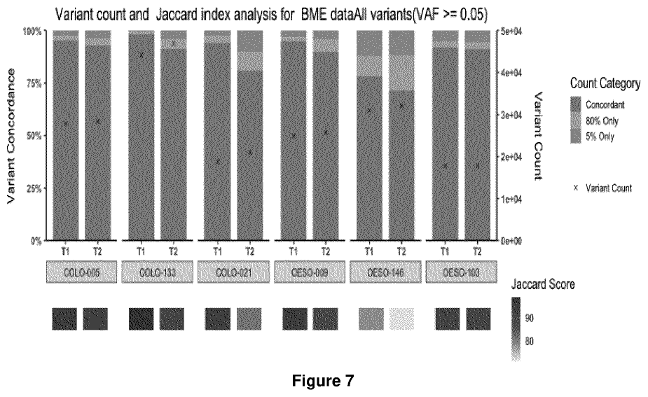

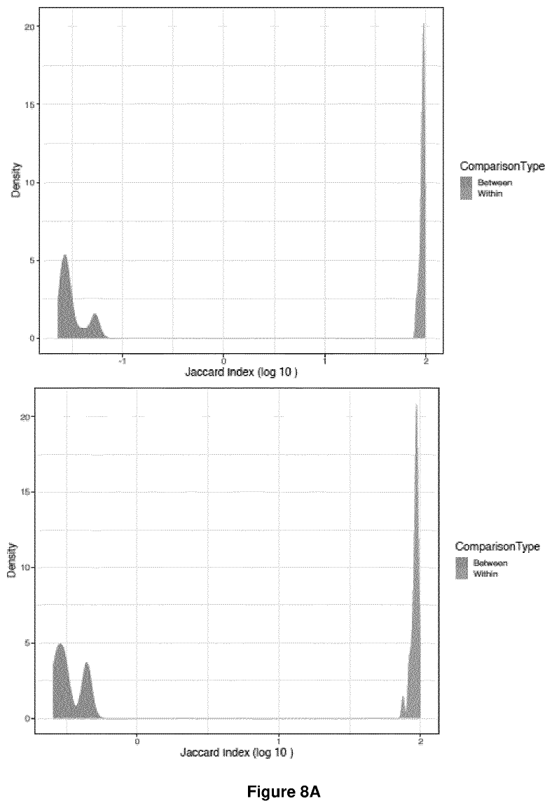

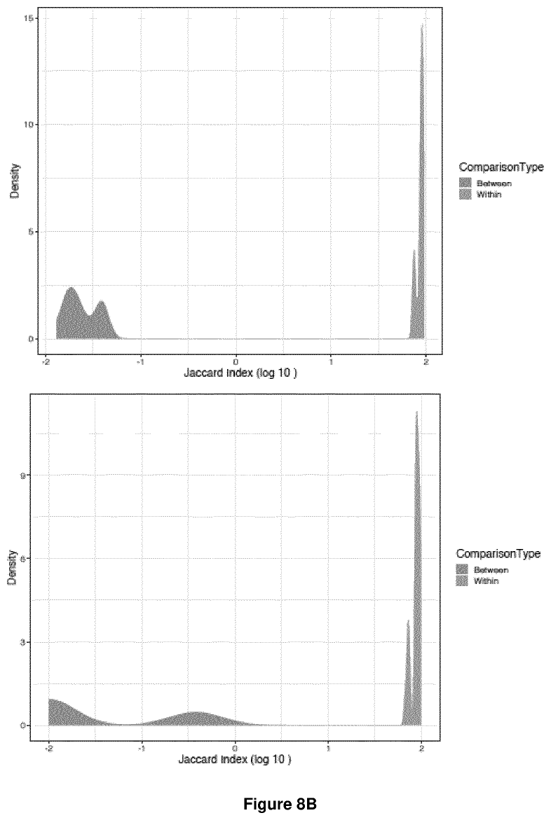

As can be seen on FIG. 5 (which shows the average number of mutations per million bases, for each time point), the total mutational burden across the six models remained fairly consistent over the six month timeframe, indicating that the 5% ECM condition does not adversely affect the acquisition of mutations during prolonged culture. A comparison of the distribution of mutations following six months in culture in 5% ECM vs. standard culture conditions indicated no change in the distribution and pattern of mutations across the genome based on culture condition (see FIG. 6 which show circos plots for 3 exemplary colon lines and 3 exemplary oesophagal lines, where the outer 3 tracks show the distribution of variants across the genome at TO 80% ECM(outer), T2 5% ECM (second from outside) and T2 80% ECM (third from outside), and the inner 3 tracks show the log R copy number (log 2(observed probe intensity/reference probe intensity)) across the genome at TO 80% ECM (fourth track from outside, third from centre), T2 5% ECM (second track from centre) and T2 80% ECM (innermost track)). Further, a very high concordance of identified variants and indels (insertions and deletions) was observed in each culture condition at a given time point, as shown on FIG. 7 which shows (top) the percentages of concordant (i.e. present in both 80% BME and 5% BME culture conditions) and discordant (i.e. present in either 80% BME or 5% BME culture conditions) mutations with a VAF of greater than 0.05, at T1 and T2 for 3 exemplary colon lines and 3 exemplary oesophagal lines, and (bottom) the Jaccard score (size of the intersection divided by the size of the union of the sets of mutations in the different conditions) for all samples of each organoid line and time point (e.g. number of mutations found in all samples of the colo-005 line at T1 divided by total number of individual mutations found across samples of the colo-005 line at T1). In 4 out of 6 models concordance was greater than 90% at both time points and show high similarity Jaccard scores (see FIG. 7). FIG. 8 shows density plots for the Jaccard scores calculated within models (high peak) and between models (loew peaks) for colon samples over all variants (top left), and over non-synonymous variants (top right), and for oesophagal samples over all variants (bottom left), and over non-synonymous variants (bottom right). Taken together this data indicates that the cultures do not show any significant global mutational changes when cultured in the alternative 5% ECM condition. There were a small number of mutations in all models and at all timepoints that were exclusive to both 5% and 80% culture conditions. None of these mutations were in cancer driver genes and could represent sequencing artefacts, variants lost in a condition due to stringent selection criteria or the acquisition of mutations due to different mutational processes operative in each of the culture conditions.

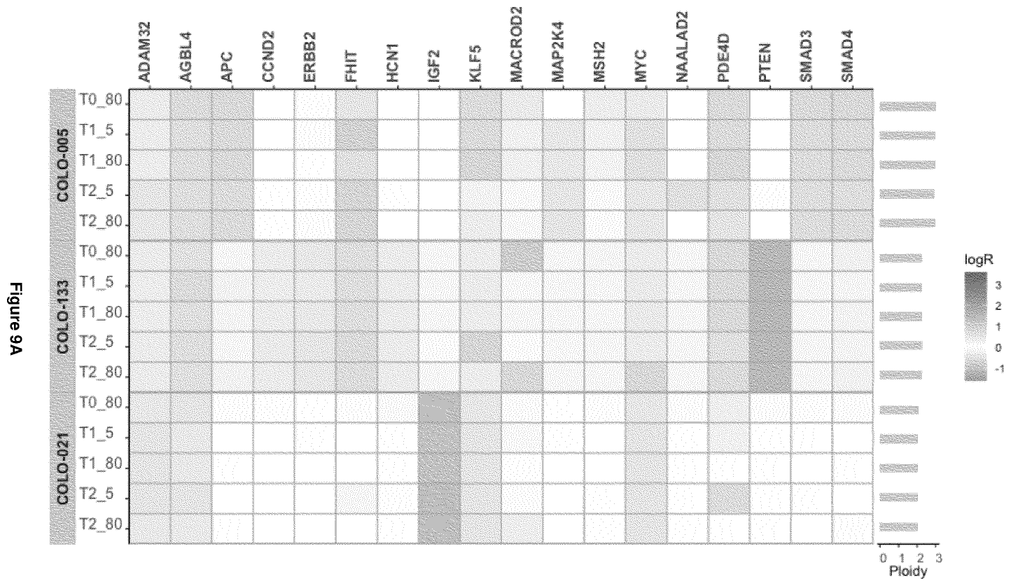

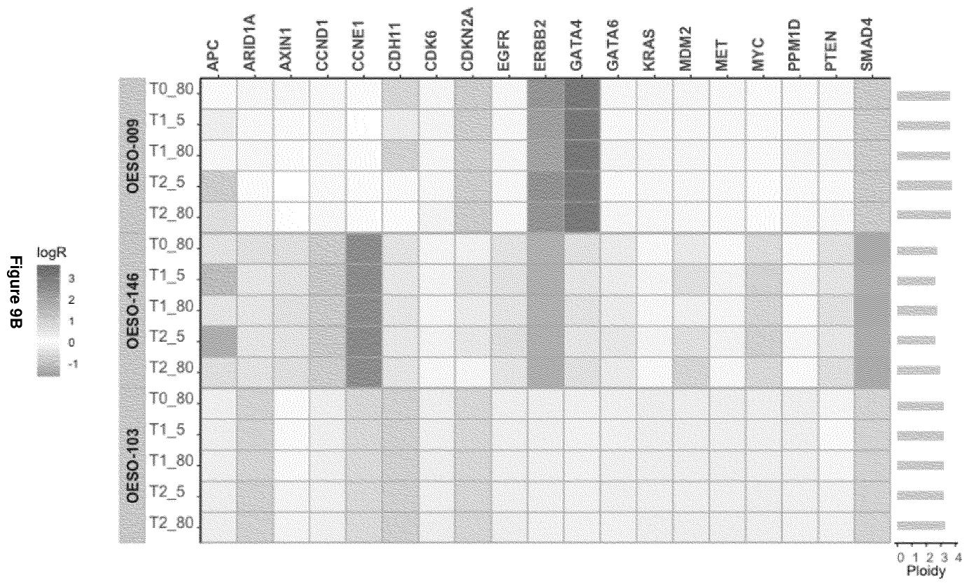

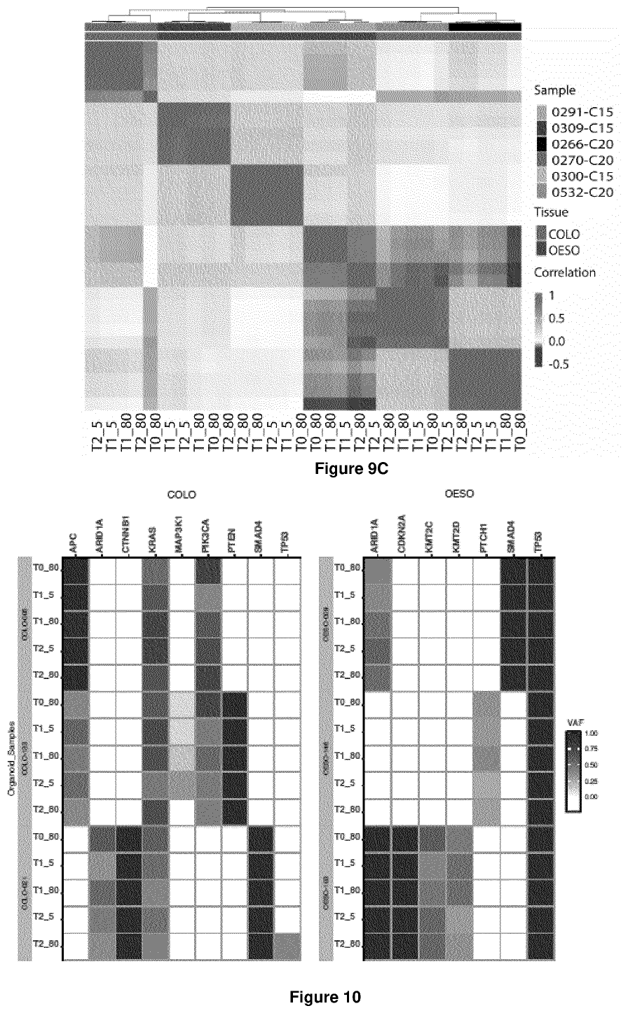

Using the mean log R copy number of all genes, the inventors observed a high correlation within samples from each model (FIG. 9C). Furthermore, the global pattern of copy number alterations at TO and T2 was shown to be consistent across time points in both culture conditions. A global overview of copy number for all models at TO and T2 in both culture conditions is shown in FIG. 6. These circos plots demonstrate that there are very few changes observed between TO and T2 in either culture condition, however there are some changes e.g. OESO-103 Chromosome 6 (which is known to have undergone chromothrypsis). To further interrogate the copy number profiles of the models across time points, the inventors identified a list of high confidence copy number altered genes in both colon and oesophagal cancer and interrogated these genes specifically (6)(7) by assessing their copy number profiles across the models and time points. The results of this analysis are shown on FIGS. 9A (for the colon cancer organoids) and 9B (for the oesophagal organoids). The data on FIGS. 9A and 9B shows that copy number alterations in these genes are for the most part consistent between the 5% BME and 80% BME culture conditions. Some alterations are acquired over time in either the 5% or 80% conditions, but the 5% condition did not show a particularly high propensity to acquire such alterations compared to the 80% condition. Importantly, the ploidy remained consistent across all conditions from a particular model, and when focusing on tissue-specific copy number driven cancer genes the vast majority remain unchanged at all time points and culture conditions (FIGS. 9A and 9B).

The inventors next focused on the presence of mutations in cancer driver genes known to have a pathogenic role in the respective cancer types. A further analysis specifically on the landscape of cancer driver mutations confirmed that the models were not accumulating additional, tissue-specific cancer driver mutations and the variant allele fraction for the driver events remained consistent over time in prolonged culture in both standard conditions and 5% ECM (see FIG. 10 which shows a heatmap of VAF for cancer driver events in the colon samples (left) and the oesophagal samples (right)). For example, KRAS mutations were observed in all colon models under both conditions and time-points, and as expected the oesophageal adenocarcinoma models all have TP53 mutations irrespective of growth conditions. There was 1 example where a missense driver mutation in TP53 (17:7577548:C:T, VAF 0.41) was acquired in COLO-021-T2-80% (HCM-SANG-0270-C20-T2 under standard conditions). However, this acquisition was identified in the model cultured under standard conditions and not 5% ECM. This most likely reflects the selection of a rare sub-clone present at TO but below detection. Interestingly, this model has previously been shown to have a transiently detected missense mutation in TP53 (17:7578478:G:T) in earlier and much deeper sequencing. It is also noted that there is a loss of MAP3K1 in the KRAS mutant COLO-133 at T2 in 80% BME, which is retained in the T2 5% sample (see FIG. 10). However, other than these two identified changes, which were observed in the standard culture condition, the driver landscape remains stable over the extended culture period. Taken together these data indicate that the cultures do not have any significant global mutational alterations or divergence in the representation of driver mutations when cultured in 5% ECM.

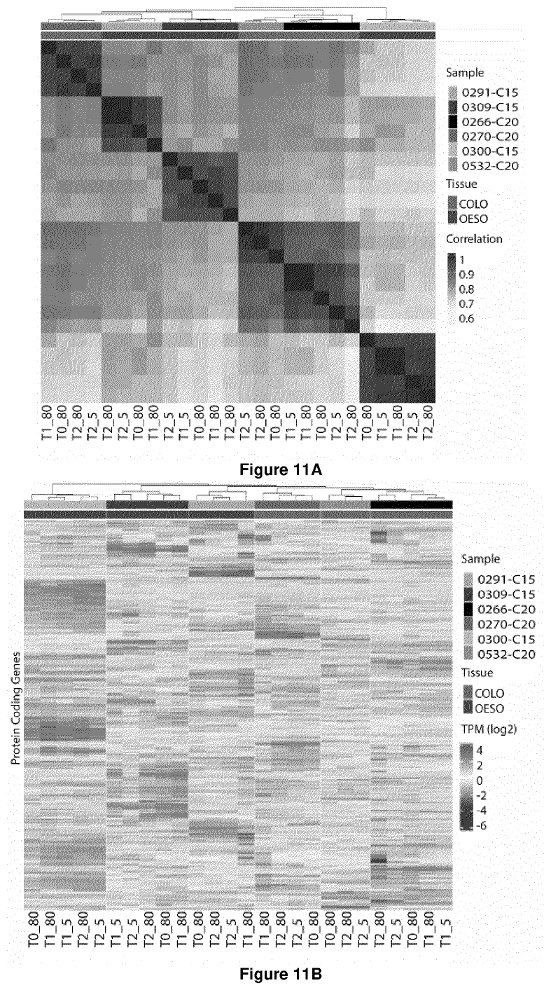

RNAseq was performed on all samples at TO, T1 and T2; unsupervised hierarchical clustering of all samples showed that at the gene expression level samples clustered first by organoid model, and then by time point (see FIG. 11A). Unsupervised hierarchical clustering of the 3,000 most differentially expressed genes demonstrated that, in the majority of models, samples clustered first by organoid model, and then by time point (FIG. 11B). This data indicates that the time point at which the RNA was harvested has more impact on the gene expression profiles of these models than the culture condition.

Together these results show that models cultured in 5% ECM are genetically and transcriptionally equivalent and virtually indistinguishable to those cultured in 80% ECM droplets.

### Example 3: Phenotypic Characterisation of Organoids in Suspension Culture—Drug Screening

Drug sensitivity testing is an important application in cancer drug development and so the inventors sought to determine whether long term culture in 5% ECM conditions led to changes in sensitivity to drug treatment. Thus, the sensitivity of four organoid models (two colon and two oesophagal lines) to 72 anti-cancer drugs was compared after propagation for up to six-months (T2) in standard or 5% ECM cultures conditions. Drug sensitivity was measured as 1—area under the dose response curve (1−AUC) determined from a 7-point dose response curve encompassing a 1,000-fold concentration range. The compounds assayed included FDA-approved drugs, compounds in clinical development, investigational compounds to a wide range of oncology targets, and also included chemotherapeutic agents. The purpose of this drug screening was to determine if long-term 5% ECM culture conditions affect organoid response to drug treatment, and was performed following previously established protocols (8, 15, 30, 31).

The assays were performed in biological triplicate and viability following treatment with the 72 drugs was averaged. Both IC50 and AUC values calculated. As shown on FIG. 12A, the correlation of the 1-AUC response in 5% vs 80% BME, showed a Pearson correlation of greater than 0.94 for each of the 4 different organoid models. Thus, the sensitivity to the drugs of the four organoids grown for 6 months in 5% or 80% ECM conditions were highly correlated. This indicates that regardless of the way in which the organoids were cultured for 6 months prior to screening, their response to drug is stable.

An analysis of variance (ANOVA) was performed to systematically identify whether the model or culture condition had the greatest impact on sensitivity of the organoids to each of the 72 individual drugs. The organoid model had a significant effect on the sensitivity to many drugs (n=54 drugs at p-value threshold 0.01), whereas for nearly all drugs the culture condition had no significant impact (FIG. 12B). The only exception was the S6K1 inhibitor PF4708671, where 5% ECM was significantly associated with reduced sensitivity in HCM-SANG-0532-C20. However, this significance is marginal and was only observed in a single model suggesting this might be due to an outlier data point.

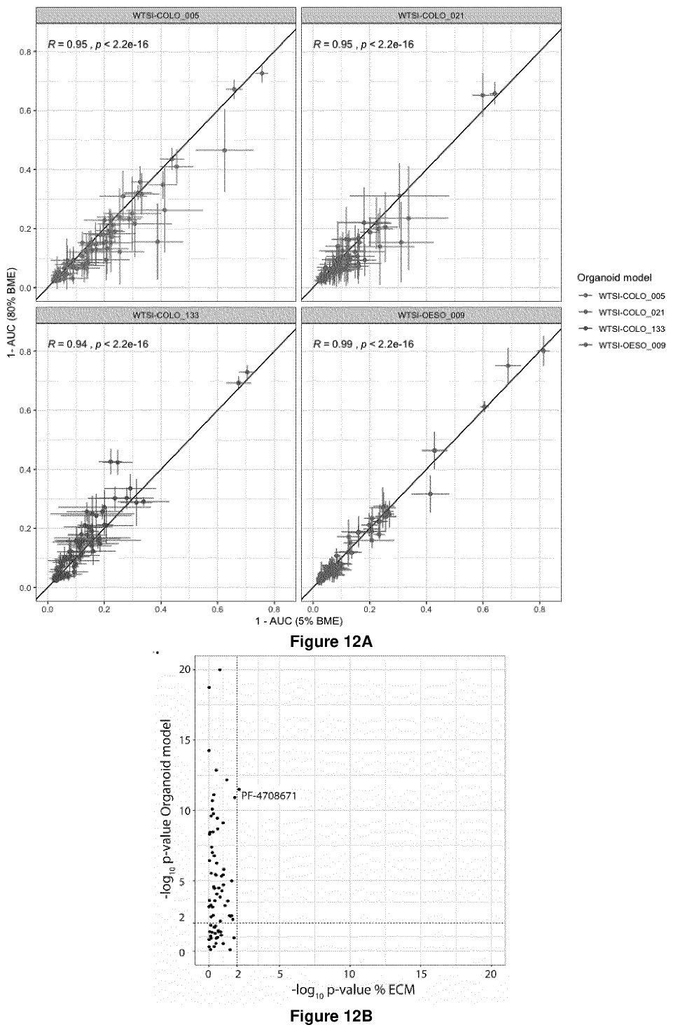

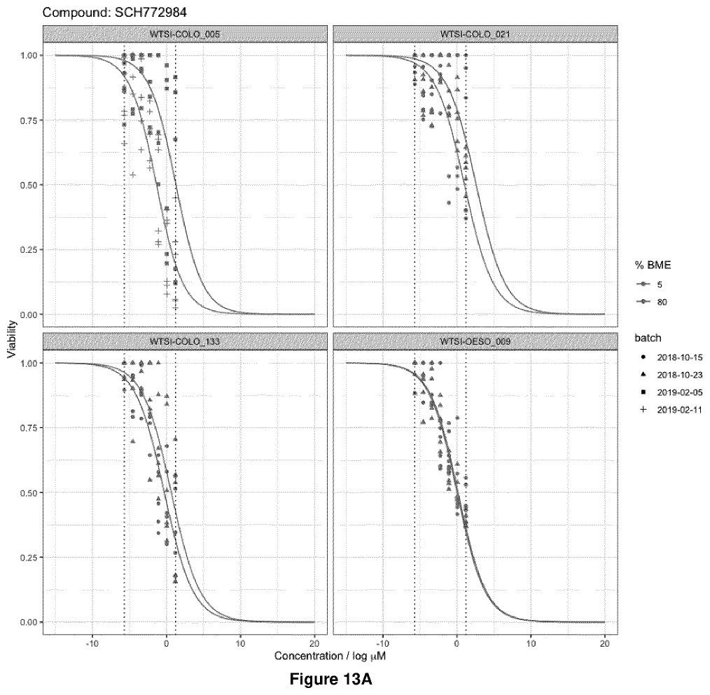

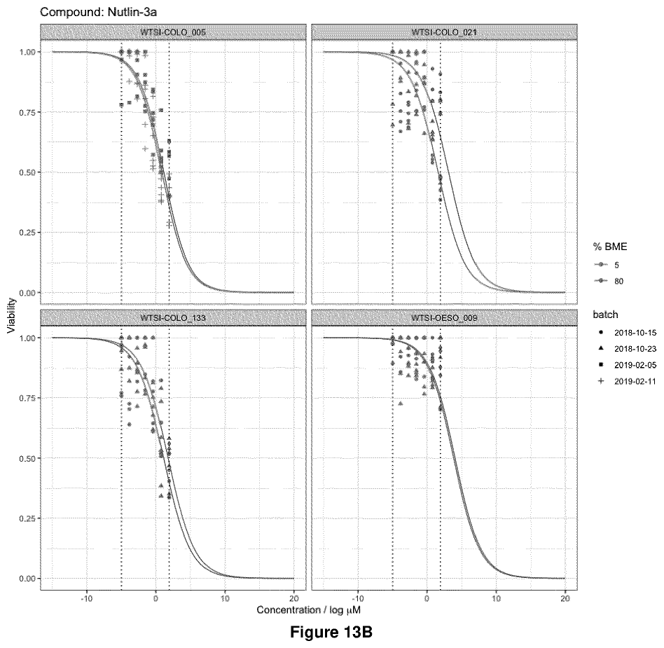

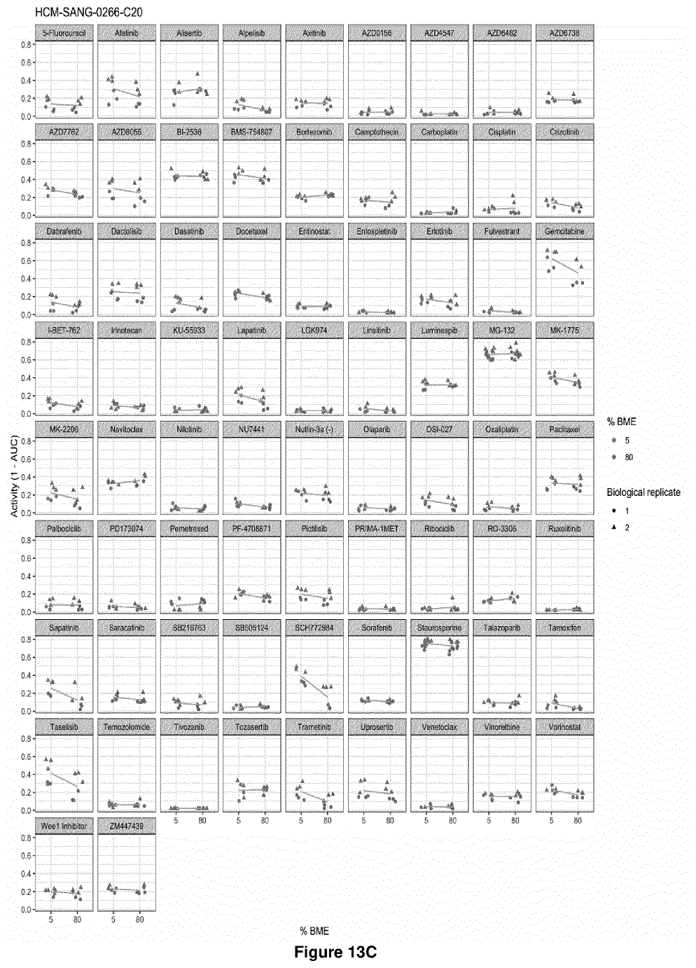

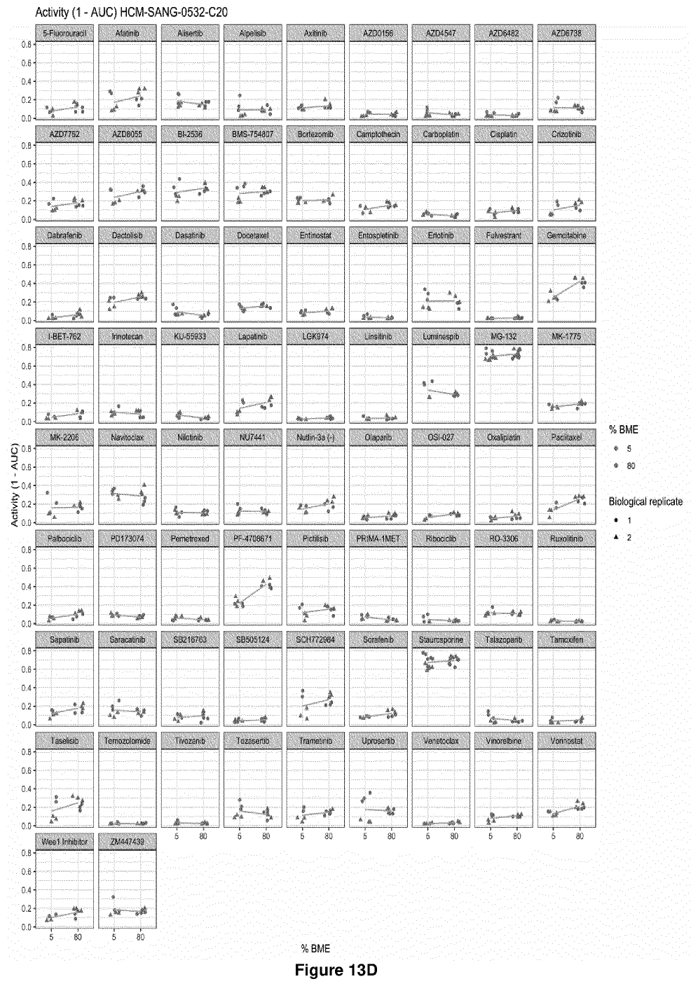

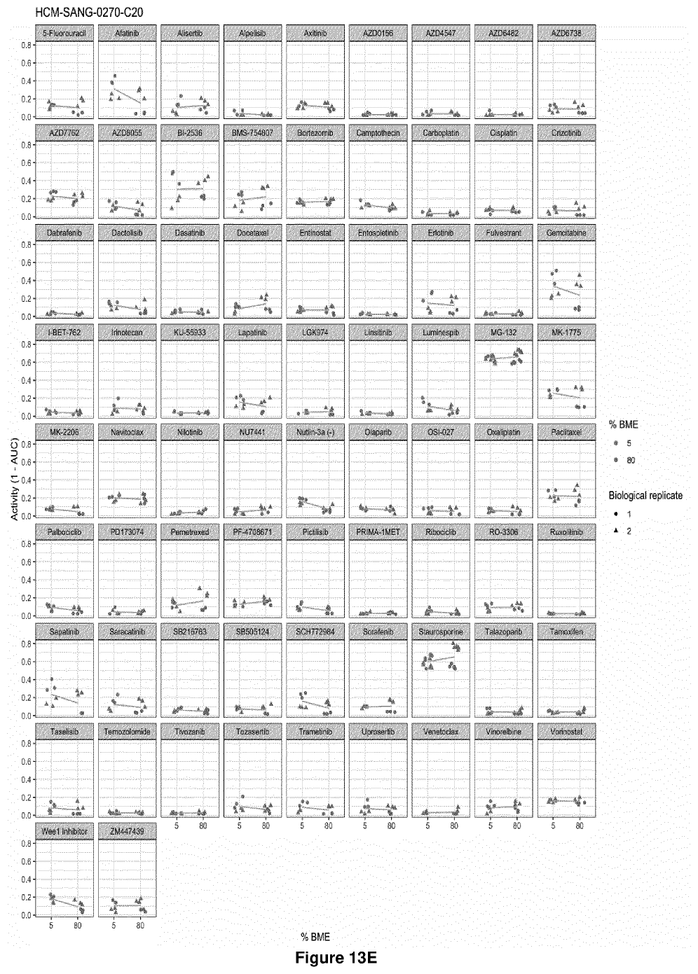

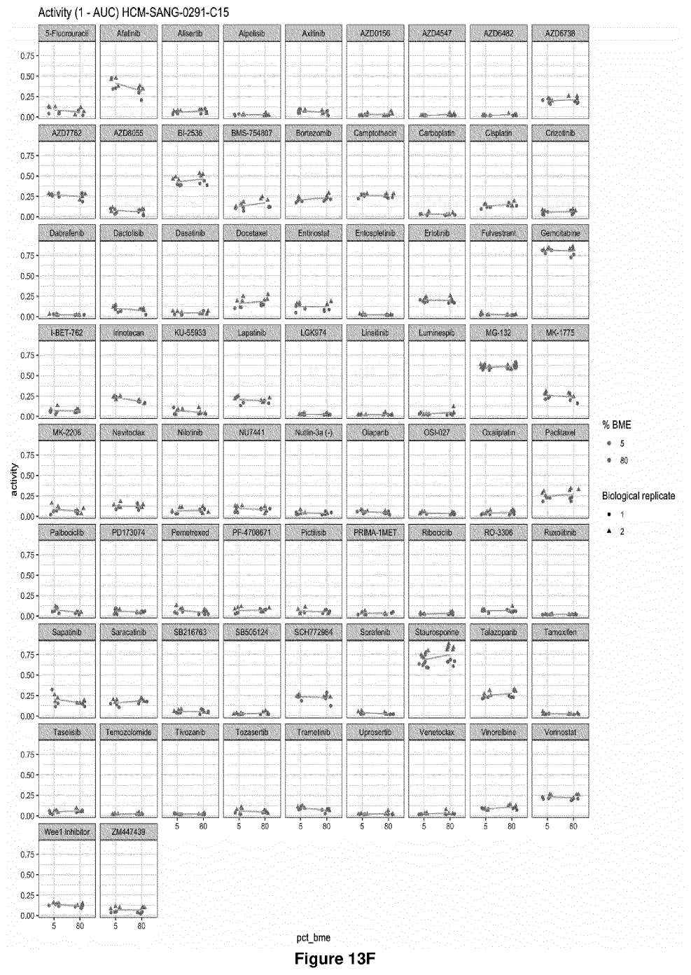

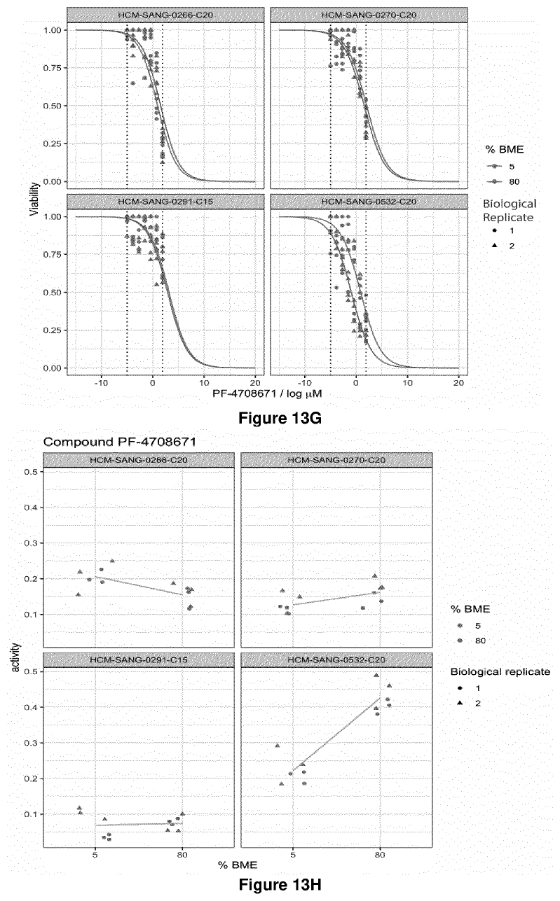

One colon model showed an outlier behaviour in response to drug SCH772984. Representative dose response curves for the four organoid models when treated with nutlin or SCH772984 are shown on FIGS. 13A-13B. This figure shows all 3 biological and 3 technical replicates, and the fitted dose response curves. This data shows tight concordance for nutlin (FIG. 13B), and for SCH772984 in the oesophageal model (bottom right plot, FIG. 13A) and two of the three colon models (FIG. 13A, top right and bottom left plots—the top left plot corresponding to COL0005, the colon organoid line for which the SCH772984 response differed between the 5% and 80% conditions). This was verified by comparison between results obtained using the COL0005 at two different dates (two different technical replicates). As shown on FIG. 13C, the results for all drugs apart from SCH772984 were consistent between the two technical replicates (represented respectively with triangles and circles) and between the 5% and 80% BME conditions. However, the data shows that one of the 90% BME technical replicates did not respond to SCH772984. This confirms that the difference seen above was likely due to technical problems, and confirms that the organoids in suspension culture show a similar phenotype in terms of drug response, compared to those in the conventional BME domes culture.

As expected, known biomarkers of drug sensitivity were observed in organoid models cultured in both conditions. For example, a TP53 wild-type colon cancer organoid HCM-SANG-0266-C20 (WTSI-COLO 005) was sensitive to the MDM2 inhibitor Nutlin-3a, whereas a TP53 mutant oesophageal organoid HCM-SANG-0291-C15 (WTSI-OESO 009) exhibited little to no response to treatment (FIG. 13B). All ERAS mutant colon cancer models were resistant to EGFR inhibition (FIGS. 13D-F). We observed differential sensitivity to the ERK inhibitor SCH77298 in HCM-SANG-0266-C20 (WTSI-COLO 005) due to culture conditions, which appeared to be the result of a single outlier replicate data point in the 80% ECM condition (FIG. 13A).

Taken together, these data indicate that long-term culturing in 5% ECM conditions prior to screening does not impact on the response of organoids models to a wide range of different anti-cancer drugs.

### Example 4: Phenotypic Characterisation of Organoids in Suspension Culture—CRISPR Drop Out Screening

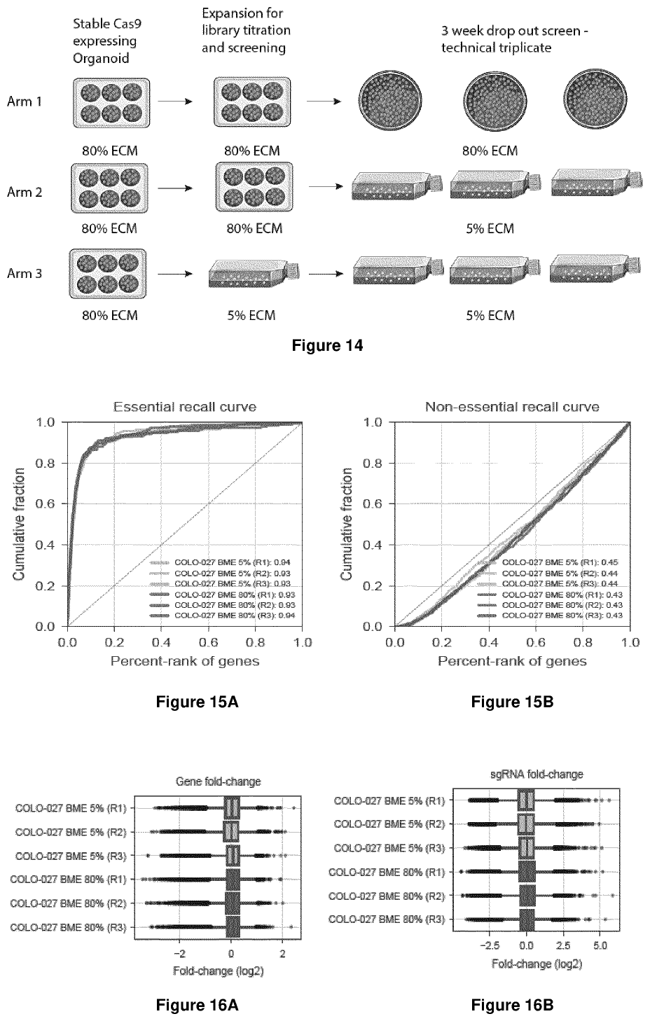

Another important application in oncology is the use of genetic perturbation approaches, such as loss-of-function CRISPR-Cas9 screens, to identify dependencies across tumour types. To further investigate whether phenotypic response is stable in 5% ECM conditions, a whole-genome CRISPR-Cas9 knockout screen targeting 18,009 genes (Yusa v1.1 library, Behan et al. (9)) was performed in COLO-027 (colorectal cancer organoid model HCM-SANG-0273-C18, in both 80% and 5% ECM. To perform a whole genome CRISPR screen in a model at 100× coverage of a 100,000 gRNA library, there is a requirement for 1×10\hat{ }8 single cells expressing Cas9 at library transduction. To minimise variation, prior to transduction with the sgRNA library the organoids were cultured under standard 80% conditions, and following sgRNA transduction they were cultured for 3 weeks to allow drop-out of sgRNAs targeting fitness genes in either 5% or 80% ECM conditions (arm 1 and arm 2 respectively, FIG. 14).

The resulting data was compared from both conditions, firstly to ascertain whether there was any variation in our ability to correctly identify known essential genes and secondly to ensure there was no systematic loss of non-essential genes from the gRNA library.

As a measure of screen quality, the recall of known essential genes (area under the recall curve (AROC)=0.93-0.94) (see FIG. 15A) was similar for both conditions, and the AROC values are equivalent to what is typically observed for screens performed in 2D cancer cell lines (9). Consistently, both sgRNA and gene level fold changes do not display any general shift in their distributions and are centred on 0 (as shown on FIG. 16A for the gene fold change and 16B for the sgRNA fold change), indicating that coverage of the library at the gRNA and gene level was maintained in both culture conditions, confirming no unexpected loss of transduced cells during the three-week assay. Further, the inventors observed positive significant correlations between all COLO-027 samples (see FIG. 17), further demonstrating that the loss of essential genes is consistent across all replicates regardless of culture condition. Notably, there was a strong positive correlation between gene log-fold change values at both conditions, confirming that the identified fitness genes were consistent between culture conditions (Pearson correlation R=0.65) (FIG. 17A and Figure S17B).

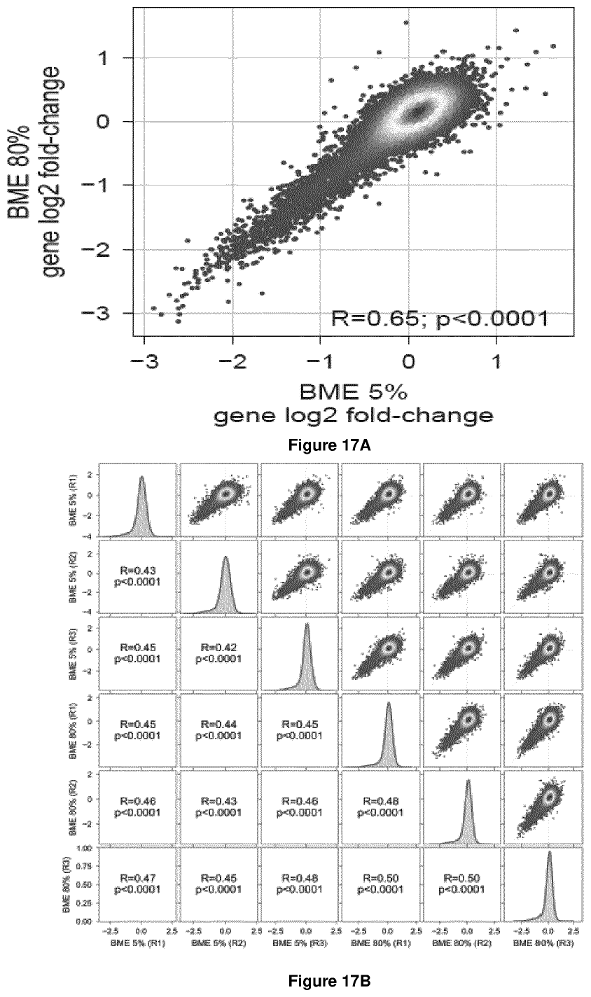

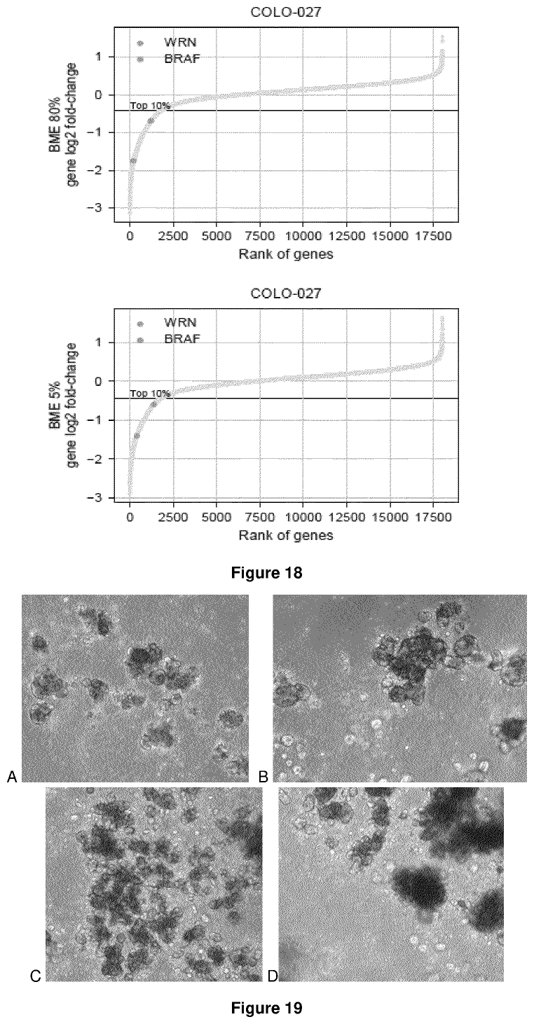

The inventors were additionally able to confirm known gene dependencies in HCM-SANG-0273-C18 (labelled as “COLO-027”). COLO-027 organoid is a microsatellite instable (MSI) cancer model and harbours BRAF constitutive activating mutation V600E. As expected, knockout of BRAF led to a strong loss of viability in both 80% and 5% conditions (top 10% dependencies, FIG. 18). As expected, the inventors found that knockout of BRAF led to a strong loss of viability both in 80% and 5% conditions (top 10% dependencies, see FIG. 18). In particular knockout of the WRN in this model also led to loss of viability in both conditions in keeping with its recent synthetic lethal association with microsatellite instability (9).

Taken together, this approach demonstrated that when taking an organoid which stably expresses Cas9 endonuclease and applying a genome wide gRNA library the resulting data is consistent irrespective of how the model is cultured. Based on this data, our organoid CRISPR screening pipeline routinely uses 5% ECM culture conditions to expand stable Cas9 expressing organoid lines and perform whole genome CRISPR drop out screens (arm 3, FIG. 14). This strategy has been adopted to maximise the benefit of working at scale with this suspension culture technique.

This data indicates that CRISPR-Cas9 knockout screens can be performed in 5% ECM conditions without loss of signal while reducing the cost and importantly making these screens practical, especially when large numbers of models are required to be screened.

### Example 5—Low ECM Concentration Supports Growth of Organoids in Suspension Culture

Having established that reducing the ECM concentration to 5% enables successfully growth of organoids in a suspension-like culture, the inventors set out to test whether this concentration could be further lowered without the significant loss of cell viability observed at 0% ECM.

In a first approach, COLO 167 organoids were seeded directly into 2.5% BME and 5% (control) in parallel and growth was assessed over 7 days for both conditions. The results of this experiment are provided in Table 6 and FIG. 19A-B (which show a similar morphology for organoids in the 2.5% (FIG. 19A) and 5% (FIG. 19B) BME conditions).

TABLE 6
Cell count and viability in 2.5% and 5% BME conditions.

| | | |
|---|---|---|
| | direct 2.5% | 5% BME |
| Treatment | seed | (+CONTROL) |

Count

| | | |
|---|---|---|
| Count 1 (per/ml) | 2.33E+05 | 2.61E+05 |
| Count 2 (per/ml) | 2.36E+05 | 2.49E+05 |
| Avg Count | 2.35E+05 | 2.55E+05 |
| STDEV | 1.50E+03 | 6.00E+03 |

Viability

| | | |
|---|---|---|
| Viability count 1 | 83.7 | 87.1 |
| Viability count 2 | 87.80 | 86.40 |
| Avg Viability | 85.75 | 86.75 |
| STDEV | 2.899137803 | 0.494974747 |

Total cell count (Suspended in 10 ml)

| | | |
|---|---|---|
| Cell count (7 days | 2.35E+06 | 2.55E+06 |
| post plate) | | |
| Starting cell count | 8.00E+05 | 8.00E+05 |

The organoids were seeded at 800,000 cells per well and by the end of the experiment there are well over 2,000,000 cells in both the 2.5% and the 5% BME conditions. A slight difference in cell count is observed but this may be simply due to the lack of replication. There was no significant loss of viability. Thus, this experiment shows that the 2.5% conditions support the growth of cancer organoid models and has no detrimental effect on cell viability in comparison to the 5% condition.

In a second approach, instead of directly seeding the organoids into 2.5% conditions, PANC 067 organoids were seeded in 5% BME and following 30 minutes more culture media was added to give a final concentration of 2.5% (a condition referred to below as 5% BME). These were also compared to a 5% BME parallel control. The results of this experiment are provided in Table 7 and FIG. 19C-D (which show a similar morphology for organoids in the 2.5% (FIG. 19C) and 5% (FIG. 19D) BME conditions).

TABLE 7
Cell count and viability in ½ 5% (2.5%) and 5% BME conditions.

| | | | | |
|---|---|---|---|---|
| | | | 5% BME | 5% BME |
| | ½ BME | ½ BME | (+CONTROL) | (+CONTROL) |
| Treatment | R1 - 2 step | R2 - 2 step | R1 | R2 |

Count

| | | | | |
|---|---|---|---|---|
| Count 1 | 1.05E+06 | 1.06E+06 | 9.27E+05 | 8.95E+05 |
| (per/ml) | | | | |
| Count 2 | 9.56E+05 | 1.03E+06 | 7.95E+05 | 8.55E+05 |
| (per/ml) | | | | |
| Count 3 | | | 7.62E+05 | |
| (per/ml) | | | | |
| Avg Count | 1.003E+06 | 1.05E+06 | 8.28E+05 | 8.75E+05 |
| STDEV | 4.70E+04 | 1.50E+04 | 6.60E+04 | 2.00E+04 |

| | | |
|---|---|---|
| Combined | Avg: 1.02E+06 | Avg: 8.52E+05 |
| replicates | STDEV: 2.97E+04 | STDEV: 3.32E+04 |
| count (per/ml) | | |

Viability

| Viability 1 | 91.2 | 92.3 | 91 | 90.3 |
|---|---|---|---|---|
| Viability 2 | 90 | 94.1 | 88.5 | 91.6 |
| Avg Viability | 90.6 | 93.2 | 89.75 | 90.95 |
| STDEV | 0.6 | 0.9 | 1.25 | 0.65 |
| Combined | Avg: 91.90 | | Avg: 90.35 | |
| replicates | STDEV: 1.84E+00 | | STDEV: 8.49E−01 | |
| viability | | | | |
| Total cell | 1.01E+07 | 1.06E+07 | 8.36E+06 | 8.84E+06 |
| count | | | | |

| | | |
|---|---|---|
| Combined | Avg: 1.03E+07 | Avg: 8.60E+06 |
| replicates | STDEV: 3.00E+05 | STDEV: 3.36E+05 |
| total cell | | |
| count | | |

R1 = replicate 1.
R2 = replicate 2.
½ BME = 2.5% BME.

Again this data shows that there is no negative effect on viability in the 2.5% BME condition, and the cells grow equally well to their 5% counterparts.

Thus, the data demonstrates that concentrations as low as 2.5% BME can also sustain organoid growth with no loss of viability.

### EXAMPLES 1-5: CONCLUSIONS

The present inventors have developed a culture methodology that increases the efficiency of growing organoid cultures at scale while retaining genomic and phenotypic stability relative to standard organoid culture conditions. Notably, the inventors extensively benchmarked the approach using multiple cancer organoid cultures from three different cancer types, monitoring global and disease relevant molecular changes and phenotypes including drug sensitivity and genetic dependencies. Moreover, the inventors provide direct evidence that it is possible to readily propagate organoid cultures in sufficient numbers making them suitable for systematic perturbations assays.

While it is currently possible to perform high throughput screening in organoids with the established technique of suspending organoids in ECM droplets which are then immersed in media, it is time consuming, labour intensive and ergonomically challenging. In this work, the inventors have identified a modified approach where reducing the ECM concentration to 5% (or even 2.5%) enables successfully growth of organoids in a suspension-like culture reducing the cost of ECM to approximately 30-40% of the 80% ECM equivalent.

The inventors have further moved on to investigate how this alternative technique compares with using the droplet technique (80% ECM), at a genomic and phenotypic level. They showed that while cultures have accrued additional mutations over extended cell culture, these do not appear to be functionally important in the appropriate tissue context. Further, they also show that the transcriptional landscape is consistent within organoids at each time point and culture condition over the longitudinal experiment

This new culture method provides a robust alternative technique for cost effective and easier expansion of organoids for high-throughput perturbation screens. This technique has not been tested for the derivation of organoids from patient tissue. This is because the inventors believe the benefits of this technique to be particularly important when working with large numbers of cells, which is not the case at the point of model derivation or resuscitation from frozen cryovials.

The methodology described here enables the efficient use of three dimensional organoid models for perturbation screens at reduced cost, and has the potential to expand the range of applications accessible using organoid culture models and increase their application in cancer research.

### REFERENCES

1. Sato T, Stange D E, Ferrante M, Vries R G, Van Es J H, Van den Brink S, et al. Long-term expansion of epithelial organoids from human colon, adenoma, adenocarcinoma, and Barrett's epithelium. Gastroenterology. 2011; 141(5):1762-72.
2. Bleijs M, van de Wetering M, Clevers H, Drost J. Xenograft and organoid model systems in cancer research. The EMBO journal. 2019; 38(15):e101654.
3. Lancaster M A, Huch M. Disease modelling in human organoids. Disease models & mechanisms. 2019; 12(7).
4. Lancaster M A, Renner M, Martin C A, Wenzel D, Bicknell L S, Hurles M E, et al. Cerebral organoids model human brain development and microcephaly. Nature. 2013; 501(7467):373-9.
5. Ben-David U, Siranosian B, Ha G, Tang H, Oren Y, Hinohara K, et al. Genetic and transcriptional evolution alters cancer cell line drug response. Nature. 2018; 560(7718):325-30.
6. Comprehensive molecular characterization of human colon and rectal cancer. Nature. 2012; 487(7407):330-7.
7. Frankell A M, Jammula S, Li X, Contino G, Killcoyne S, Abbas S, et al. The landscape of selection in 551 esophageal adenocarcinomas defines genomic biomarkers for the clinic. Nature genetics. 2019; 51(3):506-16.
8. Li X, Francies H E, Secrier M, Perner J, Miremadi A, Galeano-Dalmau N, et al. Organoid cultures recapitulate esophageal adenocarcinoma heterogeneity providing a model for clonality studies and precision therapeutics. Nature communications. 2018; 9(1):2983.
9. Behan F M, Iorio F, Picco G, Goncalves E, Beaver C M, Migliardi G, et al. Prioritization of cancer therapeutic targets using CRISPR-Cas9 screens. Nature. 2019; 568(7753):511-6.
10. Li H, Durbin R. Fast and accurate long-read alignment with Burrows-Wheeler transform. Bioinformatics (Oxford, England). 2010; 26(5):589-95.
11. Jones D, Raine K M, Davies H, Tarpey P S, Butler A P, Teague J W, et al. cgpCaVEManWrapper: Simple Execution of CaVEMan in Order to Detect Somatic Single Nucleotide Variants in NGS Data. Current protocols in bioinformatics. 2016; 56:15.0.1-.0.8.
12. Raine K M, Hinton J, Butler A P, Teague J W, Davies H, Tarpey P, et al. cgpPindel: Identifying Somatically Acquired Insertion and Deletion Events from Paired End Sequencing. Current protocols in bioinformatics. 2015; 52:15.7.1-.7.2.
13. Yates L R, Knappskog S, Wedge D, Farmery J H R, Gonzalez S, Martincorena I, et al. Genomic Evolution of Breast Cancer Metastasis and Relapse. Cancer cell. 2017; 32(2):169-84.e7.
14. Dobin A, Davis C A, Schlesinger F, Drenkow J, Zaleski C, Jha S, et al. STAR: ultrafast universal RNA-seq aligner. Bioinformatics (Oxford, England). 2013; 29(1):15-21.
15. van de Wetering M, Francies H E, Francis J M, Bounova G, Iorio F, Pronk A et al. Prospective derivation of a living organoid biobank of colorectal cancer patients. Cell. 2015 May 7; 161(4):933-45.
16. Baker L A, Tiriac H, Clevers H, Tuveson D A. 2016. Modeling pancreatic cancer with organoids. Trends Cancer 2:176-90
17. Merker S R, Weitz J, Stange D E. 2016. Gastrointestinal organoids: How they gut it out. Dev. Biol. 420:239-50.
18. Giobbe, G. G., Crowley, C., Luni, C. et al. Extracellular matrix hydrogel derived from decellularized tissues enables endodermal organoid culture. Nat Commun 10, 5658 (2019).
19. Kratochvil, M. J., Seymour, A. J., Li, T. L. et al. Engineered materials for organoid systems. Nat Rev Mater 4, 606-622 (2019).
20. Gjorevski, N., Sachs, N., Manfrin, A. et al. Designer matrices for intestinal stem cell and organoid culture. Nature 539, 560-564 (2016). https://doi.org/10.1038/nature20168
21. Victor Hernandez-Gordillo, Timothy Kassis, Arinola Lampejo, GiHun Choi, Mario E. Gamboa, Juan S. Gnecco, David T. Breault, Rebecca Carrier, and Linda G. Griffith. Niche-inspired synthetic matrices for epithelial organoid culture. bioRxiv. Oct. 16, 2019 https://www.biorxiv.org/content/10.1101/806919v1
22. Nicolas Broguiere, Luca Isenmann, Christian Hirt, Till Ringel, Silja Placzek, Emma Cavalli, Femke Ringnalda, Lukas Villiger, Richard Zullig, Roger Lehmann, Gerhard Rogler, Markus H. Heim, Julia Schüler, Marcy Zenobi-Wong, Gerald Schwank. Growth of Epithelial Organoids in a Defined Hydrogel. Advanced Materials, Volume 30, Issue 43. 10 Sep. 2018. https://doi.org/10.1002/adma.201801621
23. Raine K M, Van Loo P, Wedge D C, et al. ascatNgs: Identifying Somatically Acquired Copy-Number Alterations from Whole-Genome Sequencing Data. Curr Protoc Bioinformatics. 2016; 56:15.9.1-15.9.17. Published 2016 Dec. 8. doi:10.1002/cpbi.17
24. Peter Van Loo, Silje H. Nordgard, Ole Christian Lingjrde, Hege G. Russnes, Inga H. Rye, Wei Sun, Victor J. Weigman, Peter Marynen, Anders Zetterberg, Bjorn Naume, Charles M. Perou, Anne-Lise Børresen-Dale, and Vessela N. Kristensen. Proceedings of the National Academy of Sciences of the USA, 107:16910-16915, 2010.
25. Gonsalves, E. et al. Minimal genome-wide human CRISPR-Cas9 library. bioRxiv, 848895, doi:10.1101/848895 (2020).
26. Hart, T. et al. High-Resolution CRISPR Screens Reveal Fitness Genes and Genotype-Specific Cancer Liabilities. Cell 163, 1515-1526, doi:10.1016/j.cell.2015.11.015 (2015).
27. Dempster, J. M. et al. Agreement between two large pan-cancer CRISPR-Cas9 gene dependency data sets. Nature communications 10, 5817, doi:10.1038/s41467-019-13805-y (2019).
28. Robinson, M. D., McCarthy, D. J. & Smyth, G. K. edgeR: a Bioconductor package for differential expression analysis of digital gene expression data. Bioinformatics (Oxford, England) 26, 139-140, doi:10.1093/bioinformatics/btp616 (2010).
29. Vis, D. J. et al. Multilevel models improve precision and speed of IC50 estimates. Pharmacogenomics 17, 691-700, doi:10.2217/pgs.16.15 (2016).
30. Broutier, L. et al. Human primary liver cancer-derived organoid cultures for disease modeling and drug screening. Nature medicine 23, 1424-1435, doi:10.1038/nm.4438 (2017).
31. Driehuis, E. et al. Pancreatic cancer organoids recapitulate disease and allow personalized drug screening. Proceedings of the National Academy of Sciences of the United States of America 116, 26580-26590, doi:10.1073/pnas.1911273116 (2019).

All references cited herein are incorporated herein by reference in their entirety and for all purposes to the same extent as if each individual publication or patent or patent application was specifically and individually indicated to be incorporated by reference in its entirety.

1. A method for producing an expanded population of organoids in vitro comprising:
(i) providing a population of organoid progenitor cells or organoids; and
(ii) culturing the population of organoids in a composition comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml, thereby producing an expanded population of organoids.

2. The method of claim 1, comprising preparing an organoid culture by mixing organoids or organoid progenitor cells, and:
(a) a composition comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml, or
(b) a culture medium and a scaffold matrix, wherein the amounts of scaffold matrix and culture medium are such that the scaffold matrix is present in the resulting composition at a concentration that is equivalent to a concentration of between 2% (v/v) and 18% (v/v) of a complex protein hydrogel having a protein concentration between 12 and 18 mg/ml.

3. The method of claim 1 or claim 2, wherein the scaffold matrix is present in the composition at a concentration that is equivalent to a concentration of at least 2.5% (v/v), at least 3% (v/v) or at least 4% (v/v) of a complex protein hydrogel having a protein concentration between 12 and 18 mg/ml.

4. The method of any preceding claim, wherein culturing the population comprises maintaining the composition comprising the population of organoids in one or more low adherence cell culture containers, optionally wherein the low adherence cell culture containers is/are ultra-low attachment (ULA) or cell repellent (CR) cell culture containers.

5. The method of claim 4, wherein the low adherence cell culture containers are cell culture containers coated with an anti-adhesion coating, optionally wherein the anti-adhesion coating is a covalently bound hydrogel layer or a covalently bound hydrophobic polymer, such as a hydrophobic fluorinated polymer.

6. The method of any preceding claim, wherein culturing the population of organoids or organoid progenitor cells comprises culturing the population in suspension in the composition.

7. The method of any preceding claim, wherein the organoids are maintained in culture and expanded to a population comprising at least 10\hat{ }4, at least 10\hat{ }5, at least 10\hat{ }6 or at least 10\hat{ }7 individual organoids, and/or wherein the organoids are maintained in culture for at least 4 weeks, at least 6 weeks, at least 8 weeks, at least 2 months, at least 3 months, at least 4 months, at least 5 months or at least 6 months, and/or wherein the organoids are maintained in culture for at least 2 passages, at least 3 passages, at least 4 passages, at least 6 passages, at least 8 passages, at least 10 passages, at least 12 passages or at least 14 passages.

8. A composition suitable for expansion of organoids, comprising a culture medium and a scaffold matrix, wherein the scaffold matrix is present in the composition at a concentration that is equivalent to a concentration of between 2% (v/v) and 18% (v/v) of a complex protein hydrogel having a protein concentration between 12 and 18 mg/ml.

9. The method of any of claims 1 to 7 or the composition of claim 8, wherein a concentration of scaffold matrix that is equivalent to a concentration of between 2% (v/v) and 18% (v/v) of a complex protein hydrogel having a protein concentration between 12 and 18 mg/ml is a concentration that is between 0.02 and 0.225 times the concentration of scaffold matrix usable to culture organoids embedded in domes of the scaffold matrix.

10. The method of any of claim 1 to 7 or 9, or the composition of claim 8 or claim 9, wherein the scaffold matrix is present in the composition at a concentration that is equivalent to a concentration of between 3% (v/v) and 15% (v/v) of a complex protein hydrogel having a protein concentration between 12 and 18 mg/ml, or wherein the scaffold matrix is present in the composition at a concentration that is equivalent to a concentration of between 2.5% (v/v) and 15% (v/v) of a complex protein hydrogel having a protein concentration between 12 and 18 mg/ml, or wherein the scaffold matrix is present in the composition at a concentration that is between 0.03 and 0.1875 times the concentration of scaffold matrix usable to culture organoids embedded in domes of the scaffold matrix, or wherein the scaffold matrix is present in the composition at a concentration that is between 0.025 and 0.1875 times the concentration of scaffold matrix usable to culture organoids embedded in domes of the scaffold matrix.

11. The method of any of claim 1 to 7 or 9 or 10, or the composition of any of claims 8 to 10, wherein the scaffold matrix is a complex protein hydrogel.

12. The method or the composition of claim 11, wherein the complex protein hydrogel is present in the composition at a concentration that results in a protein concentration from the complex protein hydrogel of between 0.24 mg/ml and 3.24 mg/ml, preferably between 0.30 and 2.7 mg/ml or between 0.36 and 2.7 mg/ml.

13. The method of any of claims 1 to 7 or 9 to 11, or the composition of any of claims 8 to 12, wherein the scaffold matrix is a basement membrane extract, preferably a soluble form of basement membrane purified from Engelbreth-Holm-Swarm (EHS) sarcoma cells, such as Cultrex™ BME, Cultrex™ BME type 3, Cultrex™ BME type 2, or Corning™ Matrigel™.

14. The method or the composition of claim 13, wherein the scaffold matrix is Cultrex™ BME type 3 or Cultrex™ BME type 2, preferably wherein the scaffold matrix is Cultrex™ BME type 2, optionally wherein the Cultrex™ BME type 3 or Cultrex™ BME type 2 is present at a concentration of between 2% (v/v) and 18% (v/v), between 2.5% (v/v) and 18% (v/v), between 2.5% (v/v) and 15% (v/v) between 3% (v/v) and 18% (v/v), or between 3% (v/v) and 15% (v/v).

15. The method of any of claims 1 to 7 or 9 to 14, or the composition of any of claims 8 to 14, wherein the scaffold matrix is present in the composition at a concentration that is equivalent to a concentration of between 2.5% (v/v) and 18% (v/v), between 3% (v/v) and 18% (v/v), between 4% (v/v) and 18% (v/v), between 2.5% (v/v) and 15% (v/v), between 3% (v/v) and 15% (v/v), between 4% (v/v) and 15% (v/v), between 4% (v/v) and 12% (v/v), between 5% (v/v) and 10% (v/v), or about 5% (v/v) of a complex protein hydrogel having a protein concentration between 12 and 18 mg/ml, or wherein the scaffold matrix is present in the composition at a concentration that is between 0.025 and 0.225, between 0.03 and 0.225, between 0.04 and 0.1875, between 0.03 and 0.1875, between 0.025 and 0.1875, between 0.04 and 0.15, between 0.04 and 0.125, between 0.05 and 0.125, or about 0.05-0.0625 times the concentration of the scaffold matrix that is usable to culture organoids embedded in domes of the scaffold matrix.

16. The method of any of claims 1 to 7 or 9 to 15, or the composition of any of claims 8 to 15, wherein the culture medium is a chemically defined medium, and/or wherein the culture medium comprises a basal medium, preferably Advanced Dulbecco's modified eagle medium (DMEM).

17. The method of any of claims 1 to 7 or 9 to 16, or the composition of any of claims 8 to 16, wherein the culture medium comprises one or more media supplement, such as N2 (Gibco), B-27™ (ThermoFisher) and/or one or more additional supplements which may include L-glutamine or substitutes, such as L-alanyl-L-20 glutamine (e.g. Glutamax™), nicotinamide, N-acetylcysteine, buffers, such as HEPES, and antibiotics such as blasticidin or puromycin.

18. The method of any of claims 1 to 7 or 9 to 17, or the composition of any of claims 7 to 16, wherein the culture medium additionally comprise one or more compounds selected from: growth factors (such as epidermal growth factor (EGF), fibroblast growth factor 10 (FGF10)), a TGFβ inhibitor, a non-canonical Wnt signalling potentiator, a BMP inhibitor, hormones (such as e.g. gastrin and/or prostaglandin E2), a canonical Wnt ligand, and a p38 MAPK signalling inhibitor.

19. A method of passaging or changing the medium in an organoid culture, comprising:
(i) providing a cell culture comprising a population of organoids and a composition as described in any one of claims 8 to 18;
(ii) centrifuging the cell culture to obtain a pellet comprising the organoid population and a supernatant;
(iii) optionally disrupting the organoids;
(iv) mixing the (optionally disrupted) organoids with a composition as described in any one of claims 8 to 18, thereby producing a passaged or medium-changed organoid culture.

20. The method of claim 19, wherein disrupting the organoids comprises exposing the pellet comprising the organoids to a proteolytic solution, preferably for between 5 and 10 minutes, and/or mechanically disrupting the organoids, and optionally centrifuging the composition to obtain a pellet comprising the organoid population and a supernatant.

21. The method of claim 19 or claim 20, wherein centrifuging the cell culture comprises centrifuging the population at 400 g, 500 g, 600 g, 700 g, 800 g or 900 g for about 60 seconds, 90 seconds, 120 seconds, 150 seconds or 180 seconds, preferably at 800 g for 120 seconds.

22. The method of any of claims 19 to 21, further comprising dispensing the passaged or medium-changed organoid culture in one or more low adhesion cell culture containers.

23. The method of any of claims 19 to 22, wherein mixing the organoids with a composition as described in any one of claims 8 to 18 comprises mixing the organoids with a culture medium and adding a scaffold matrix to the composition comprising the organoids and the culture medium.

24. A method of screening an organoid or a population of organoids comprising: contacting an organoid or population of organoids with a test compound; and determining the effect of the test compound on the organoids or population of organoids, wherein the organoids or population of organoids were obtained using the methods of claims 1 to 7, and/or wherein the contacting is performed while the organoids are in suspension in a composition as described in any one of claims 8 to 18.

25. The method of claim 24, wherein screening comprises performing a drug screen, gene editing screen or RNA interference screen, preferably wherein screening comprises a CRISPR gene editing screen, preferably a genome-wide gene editing screen.

26. The method of any of claims 1 to 7, 9 to 18 or 19 to 25, or the composition of any of claims 8 to 18, wherein the organoids are colon, pancreas, oesophagus, breast, lung, ovary or prostate organoids.

27. The method of any of claims 1 to 7, 9 to 18 or 19 to 26, or the composition of any of claim 8 to 18 or 26, wherein the organoids are derived from primary tissue, preferably cancerous tissue.

28. The method of any of claims 1 to 7, 9 to 18 or 19 to 27, or the composition of any of claim 8 to 18, 26 or 27, wherein the organoids are derived from colon cancer tissue, pancreatic cancer tissue, oesophageal cancer tissue, breast cancer tissue, lung cancer tissue, ovary cancer tissue, or prostate cancer tissue, preferably wherein the organoids are derived from colon cancer tissue, pancreatic cancer tissue, or oesophageal cancer tissue.

29. The method of any of claims 1 to 7, 9 to 18 or 19 to 28, or the composition of any of claims 8 to 18 or 26 to 28, wherein the organoids are mammalian organoids, preferably from human or mouse.

30. A kit for the production of expanded populations of organoids comprising a composition as described in any one of claims 8 to 18 or a culture medium (or equivalent amount of concentrated medium) and a scaffold matrix in relative amounts as described in any one of claims 8 to 18.

31. The kit of claim 30, further comprising one or more low adherence cell culture containers, optionally wherein the low adherence cell culture containers is/are ultra-low attachment (ULA) or cell repellent (CR) cell culture containers.

32. The kit of claim 31, wherein the low adherence cell culture containers are cell culture containers coated with an anti-adhesion coating, optionally wherein the anti-adhesion coating is a covalently bound hydrogel layer or a covalently bound hydrophobic polymer, such as a hydrophobic fluorinated polymer.
